SEQUENCE LISTING (12 sequences)

SEQ ID NO: 1 - DNA, 21 bp, Artificial sequence
  Sequence is synthesized
gatggccttgagtatatcctg

SEQ ID NO: 2 - DNA, 21 bp, Artificial sequence
  Sequence is synthesized
cagctctggactccagcttgc

SEQ ID NO: 3 - DNA, 21 bp, Artificial sequence
  Sequence is synthesized
ggaaactatgtggctgatctc

SEQ ID NO: 4 - DNA, 21 bp, Artificial sequence
  Sequence is synthesized
gtgagagccaagatcgaggag

SEQ ID NO: 5 - DNA, 21 bp, Artificial sequence
  sequence is synthesized
tagactctgcgcgccataact

SEQ ID NO: 6 - DNA, 21 bp, Artificial sequence
  Sequence is synthesized
cactaagaaggcagaggccaa

SEQ ID NO: 7 - DNA, 21 bp, Artificial sequence
  Sequence is synthesized
cctgcatgattcctgattgga

SEQ ID NO: 8 - DNA, 18 bp, Artificial sequence
  Sequence is synthesized
gcctaagctcacctgcgg

SEQ ID NO: 9 - DNA, 21 bp, Artificial sequence
  Sequence is synthesized
caccgcgactcctgctgcttt

SEQ ID NO: 10 - DNA, 19 bp, Artificial sequence
  Sequence is synthesized
gggggttcgggcactgctt

SEQ ID NO: 11 - DNA, 24 bp, Artificial sequence
  Sequence is synthesized
caacgtcactatgcagatcatgcg

SEQ ID NO: 12 - DNA, 19 bp, Artificial sequence
  Sequence is synthesized
tcaccgccttggcttgtca

(19) United States
(12) Patent Application Publication
(10) Pub. No.: US 2008/0206243 A1
(43) Pub. Date: Aug. 28, 2008

(54) Methods for Treating Kidney Disorders

(75) Inventors: Megan Baldwin, San Francisco, CA (US); Hans-Peter Gerber, Bellevue, WA (US); Napoleone Ferrara, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/110,042

(22) Filed: Apr. 25, 2008

Related U.S. Application Data
(63) Continuation of application No. 11/691,465, filed on Mar. 26, 2007, now abandoned.
(60) Provisional application No. 60/786,246, filed on Mar. 27, 2006.

(51) Int. Cl. A61K 39/395; A61K 38/18

(52) U.S. Cl. 424/133.1; 424/158.1; 514/2; 424/141.1

(57) ABSTRACT

Provided are methods of treating kidney disorders in a subject by administering an effective amount of VEGFR agonist, e.g., a Flt1 agonist to a subject. The agonists are composed of compositions comprising VEGFR agonists, e.g., VEGF, antibodies directed to Flt1, Flt1 ligands, Flt1 small molecule activators, or Flt1 selective agents in a pharmaceutically acceptable carrier for use in activating Flt1.

### RELATED APPLICATION

This application is a continuation of U.S. Ser. No. 11/691,465, filed on Mar. 26, 2007 which claims priority to and the benefit of U.S. Provisional Application Ser. No. 60/786,246, filed Mar. 27, 2006, the specifications of which are incorporated herein in their entirety.

### FIELD OF THE INVENTION

The invention relates to therapeutic uses of VEGFR modulating agents, including methods of utilizing VEGFR agonists for treating kidney (renal) disorders.

### BACKGROUND OF THE INVENTION

Vascular endothelial growth factor (VEGF-A) regulates a variety of vascular functions, including endothelial cell differentiation and survival (see, e.g., Ferrara, N., et al. The biology of VEGF and its receptors. Nat Med 9:669-676 (2003)), via the activation of two tyrosine kinase receptors, Flt1 (VEGFR-1) and Flk1 (KDRNVEGFR-2) expressed on endothelial cells. Recent studies identified VEGF receptor expression on various non-endothelial cells, including hematopoietic stem cells, suggesting non-vascular regulatory functions for the VEGF ligand/receptor system. See, e.g., Autiero, M., et al. Placental growth factor and its receptor, vascular endothelial growth factor receptor-1: novel targets for stimulation of ischemic tissue revascularization and inhibition of angiogenic and inflammatory disorders. J Thromb Haemost 1: 1356-1370 (2003). Within the kidney, VEGF receptors are mainly found on pre-glomerular, glomerular, post-glomerular (see, e.g., Thomas, S., et. al. Vascular endothelial growth factor receptors in human mesangium in vitro and in glomerular disease. J Am Soc Nephrol 11:1236-12433 (2000)) and peritubular endothelial cells as well as on glomerular mesangial cells, but not on podocytes. See, e.g., Gruden, G., et al., 1997. Mechanical stretch induces vascular permeability factor in human mesangial cells: mechanisms of signal transduction. Proc Natl Acad Sci USA 94:12112-12116 (1997); Harper, S. J., et al. Expression of neuropilin-1 by human glomerular epithelial cells in vitro and in vivo. Clin Sci (Lond) 101:439-446 (2001); and, Takahashi, T., et al. Protein tyrosine kinases expressed in glomeruli and cultured glomerular cells: Flt-1 and VEGF expression in renal mesangial cells. Biochem Biophys Res Commun 209:218-226 (4-6) (1995). In normal adult kidney, VEGF-A expression is most prominent in glomerular podocytes and tubular epithelial cells, lower in mesangial but undetectable in endothelial cells. See, e.g., Noguchi, K., et al. Activated mesangial cells produce vascular permeability factor in early-stage mesangial proliferative glomerulonepbritis. J Am Soc Nephrol 9:1815-1825 (1998); and, Simon, M., et al. Expression of vascular endothelial growth factor and its receptors in human renal ontogenesis and in adult kidney. Am-J-Physiol 268:F240-250 issn: 0002-9513 (1995). Based on the location of expression, VEGF-A was thought to play a regulatory role in kidney homeostasis and glomerular filtration via mostly para- or juxtacrine effector functions, targeting glomerular and peritubular endothelial cells. Various studies have evaluated a role of VEGF-A during kidney development and in renal injury models. See, e.g., de Vriese, A. S. et al. Antibodies against vascular endothelial growth factor improve early renal dysfunction in experimental diabetes. J Am Soc Nephrol 12: 993-1000 (2001); Eremina, V. et al. Glomerular-specific alterations of VEGF-A expression lead to distinct congenital and acquired renal diseases. J Clin Invest 111:707-16 (2003); Kang, D. H., et al. Impaired angiogenesis in the remnant kidney model: II. Vascular endothelial growth factor administration reduces renal fibrosis and stabilizes renal function. J Am Soc Nephrol 12:1448-57 (2001); Masuda, Y. et al. Vascular endothelial growth factor enhances glomerular capillary repair and accelerates resolution of experimentally induced glomerulonephritis. Am J Pathol 159, 599-608 (2001); and, Ostendorf, T. et al. VEGF(165) mediates glomerular endothelial repair. J Clin Invest 104:913-23 (1999).

Dysregulation of VEGF-A is a common feature in experimental models of renal diseases, including tumors, diabetes, and glomerulonephritis (see, e.g., Khamaisi, M., et al. The emerging role of VEGF in diabetic kidney disease. Nephrol Dial Transplant 18:1427-1430 (2003); and, Schrijvers, B. F., et al. The role of vascular endothelial growth factor (VEGF) in renal pathophysiology. Kidney Int 65:2003-2017 (2004)). VEGF-A and its receptors are up-regulated in experimental animals or humans with type 1 and type 2 diabetes at least for a certain time period, while decreased VEGF-A levels were associated with the development of glomerulosclerosis and tubulointerstitial fibrosis in remnant kidneys in a variety of progressive kidney diseases. See, e.g., Honkanen, E., et al. Decreased expression of vascular endothelial growth factor in idiopathic membranous glomerulonephritis: relationships to clinical course. Am J Kidney Dis 42:1139-1148 (2003); Korbet, S. M., et al. The racial prevalence of glomerular lesions in nephrotic adults. Am J Kidney Dis 27:647-51 (1996); Spyridis, E., et al. Platelet endothelial cell adhesion molecule-1 and angiogenic factor expression in idiopathic membranous nephropathy. Am J Kidney Dis 41:360-365 (2003); and, Srivastava, T., et al. High incidence of focal segmental glomerulosclerosis in nephrotic syndrome of childhood. Pediatr Nephrol 13:13-8 (1999). One of the main histopathologic characteristics of FSGS is the accumulation of deposits of extracellular matrix (ECM) or glomerulosclerosis. The pathogenesis of glomerulosclerosis is unknown, and it is unknown which of the three cell types present within the glomerulus (podocytes, endothelial, or mesangial cells) participate in the fibrotic process.

Mesangial cells replicate with increased production of extracellular matrix (ECM) as part of the glomerular response to renal injury, regardless of the type of injury, e.g. in glomerulonephritis or diabetic nephropathy. This process impairs glomerular ultrafiltration, resulting in glomerular sclerosis and end-stage renal failure. See, e.g., Buschhausen, L., et al. Kidney fibrosis impairs glomerular ultrafiltration and results in glomerular sclerosis and end-stage renal failure. Regulation of mesangial cell function by vasodilatory signaling molecules. Cardiovasc Res 51:463-469 (2001). Podocyte abnormalities identified in transgenic modules of glomerulosclerosis (see, e.g., Shih, N. Y., et al. Congenital nephrotic syndrome in mice lacking CD2-associated protein Science 286:312-315 (1999)) or in patients (see, e.g., Srivastava, T., et al. Synaptopodin expression in idiopathic nephrotic syndrome of childhood. Kidney Int 59:118-125 (2001)), suggest that these cells may play a role in the initiation of glomerular scarring. Other models have implicated endothelial or mesangial cells in the sclerotic process (see, e.g., Schnaper, H. W., et al. TGF-beta signal transduction and mesangial cell fibrogenesis. Am J Physiol Renal Physiol 284:F243-252 (2003)). In many models of glomerulosclerosis (as well as in FSGS clinically), ECM accumulation often appears to begin in the mesangium. All three cell-types within kidney glomeruli are associated and may in some way contribute to disease progression.

VEGF-A is thought to have a functional role on mesangial cells based on studies that showed increased proliferation of primary human mesangial cells in response to VEGF stimulation (Onozaki, A., et al. Rapid change of glucose concentration promotes mesangial cell proliferation via VEGF: inhibitory effects of thiazolidinedione. Biochem Biophys Res Commun 317:24-29 (2004)), induction of collagen synthesis (Amemiya, T., et al. Vascular endothelial growth factor activates MAP kinase and enhances collagen synthesis in human mesangial cells. Kidney Int 56:2055-2063 (1999)) and increased nitric oxide production (Trachtman, H., et al. Effect of vascular endothelial growth factor on nitric oxide production by cultured rat mesangial cells. Biochem Biophys Res Commun 245:443-446 (1998)). See also, e.g., Thomas, S., et al. Vascular endothelial growth factor receptors in human mesangium in vitro and in glomerular disease. J Am Soc Nephrol 11: 1236-1243 (2000). However, despite the responsiveness of mesangial cells to VEGF-A stimulation in vitro, the nature of the VEGF receptor(s) involved and the effect of alterations in VEGF-A production in mesangial cells during kidney development remained unknown.

There is a need to discover and understand the biological functions of VEGF and VEGF receptors in the kidney and in the cell types of the kidney. Understanding the biological function of these molecules can lead to treatments for kidney diseases. The invention addresses these and other needs, as will be apparent upon review of the following disclosure.

### SUMMARY OF THE INVENTION

The invention provides methods for treating renal disease in a subject. For example, a method of the invention comprises administering to the subject with renal disease an effective amount of a VEGFR modulating agent. The VEGFR modulating agent useful for the invention includes, but is not limited to, e.g., an agonist specific to at least one or more of the VEGF receptors such as a VEGF, VEGFR-1 (Flt-1) agonist, a Flt-1 selective VEGF variant (Flt-sel) that selectively binds to Flt-1, a growth factor that binds and activates Flt-1 such as PIGF or VEGF-B, an anti-VEGFR-1 agonistic antibody (e.g., monoclonal, polyclonal, antibody fragment, a human, humanized or chimeric antibody), a small molecule Flt1 agonist, etc. In one embodiment of the invention, the VEGFR modulator is a Flt1 agonist. In one embodiment, the VEGFR-1 agonist is administered in combination with an angiogenic agent, e.g., VEGF, an additional VEGFR1 ligand or agonist, VEGFR2 ligand, a VEGFR-2 (KDR) selective variant thereof, an anti-VEGFR-2 agonist antibody, VEGF-C, VEGF-D, a growth factor that binds and activates VEGFR1 and/VEGFR2, etc. Kidney diseases that can be treated by the invention include, but are not limited to, inflammatory kidney disease (e.g., characterized by alterations in inflammatory cells, immune complex depositions (e.g., IgM deposition), complement activation (e.g., activation of C1q, C3 and C4) or a combination thereof), nephritis, glomerulosclerosis, glomerulonephritis (renal failure) (e.g., which can be determined by proteinuria, glomerular sclerosis, hypertension, decreased survival of kidney mesangial cells, an increase in gene expression of ECM synthesis, a reduction in matrix degradation and/or a combination of these factors), focal segmental glomerulosclerosis (FSGS), etc. In certain embodiments of the invention, the subject has an infection that results in renal disease.

In certain embodiments, the renal disease is characterized by a decrease in VEGF levels. In certain embodiments of the invention, the disease comprises alterations in the cell types of the kidney (e.g., mesangial cells, podocyte, and/or endothelial cells).

In certain embodiments of the invention, the agent of the invention, which is delivered to the subject, is a protein or polypeptide. In certain embodiments of the invention, an agent of the invention can be administered to the subject through a systemic delivery system. The systemic delivery system can comprise a slow release preparation comprising agent, e.g., purified agent, and a polymer matrix. In one embodiment, a cell preparation comprising mammalian cells (e.g., CHO cells) expressing a recombinant form of the agent is administered. Alternatively, the subject agent of the invention can be administered via a kidney-targeted gene delivery vector comprising a nucleic acid encoding the agent. Well established viral or nonviral vectors for gene therapy can be used, e.g., a kidney-targeted gene delivery vector.

An article of manufacture and a kit comprising a VEGFR modulating agent are also provided, as well diagnostic kits and methods

### BRIEF DESCRIPTION OF THE FIGURES

FIG. 1, Panels a-h, illustrate characterization of Flt1-Cre transgenic mice using the ROSA26 LacZ reporter strain and generation of Flt1-Cre; VEGF-loxP mice. (a) Genotype frequency of offspring arising from crosses of Flt1-Cre⁺; VEGF^{(loxP/+)} and Flt1-Cre⁻; VEGF^{(loxP/loxP)} mice. Mice of all resulting genotypes are born at the expected Mendelian frequency. (b) Survival curve for Flt1-Cre⁺; VEGF^{(loxP/loxP)} mice. Decreased survival is evident 4 weeks after birth, and >95% of these mice were dead by 12 weeks of age. (c) Percentage kidney to body mass ratio in Flt1-Cre⁻ compared with Flt1-Cre⁺; VEGF^{(loxP/loxP)} mice aged 7.5 weeks. The Flt1-Cre⁺; VEGF^{(loxP/loxP)} kidneys weigh significantly less than those of controls. Flt1-Cre⁻, n=25; Flt1-Cre⁺; VEGF^{(loxP/loxP)}, n=10; *p<0.0001. (d) (Right) Typical appearance of a kidney excised from a Flt1-Cre⁺; VEGF^{(loxP/loxP)} mouse at 4 weeks of age compared with (left) a WT and phenotypically normal kidney. Flt1-Cre⁺; VEGF^{(loxP/loxP)} kidneys are frequently cystic and pale in appearance. (e) (Top) Silver stain to detect protein present in the urine of 4-5 week old Flt1-Cre; VEGF-loxP mice. One μL of urine from three mice representative for each genotype shown at the top of the panel, was subjected to SDS-PAGE and silver stain. Abundant protein is detected in the urine of Flt1-Cre⁺, VEGF^{(loxP/loxP)} mice, but not in Flt1-Cre⁺, VEGF^{(loxP/+)} or Flt1-Cre⁻ mice, indicating severe proteinuria in the conditional VEGF knockouts. (Bottom) Western blot analysis of urine from Flt1-Cre; VEGF-loxP mice using an antibody raised against albumin. (f) Levels of blood urea nitrogen (B.U.N.) in Flt1-Cre; VEGF-loxP mice aged 7.5 weeks. Flt1-Cre⁺; VEGF^{(loxP/loxP)}, n=9; Flt1-Cre⁺; VEGF^{(loxP/+)}, n=8; Flt1-Cre⁻, n=16; **p value<0.0001. (g) Serum creatinine levels in 7.5 week old Flt1-Cre; VEGF-loxP mice. Flt1-Cre⁺; VEGF^{(loxP/loxP)}, n=9; Flt1-Cre⁺; VEGF^{(loxP/+)}, n=8; Flt1-Cre⁻, n=16; *p value<0.05. (h) Mean arterial blood pressure (MAP) and mean heart rate during MAP measurement. (Left axis and columns) MAP +/− standard deviation is depicted as black columns. MAP is significantly elevated in Flt1-Cre⁺; VEGF^{(loxP/loxP)} compared with Flt1-Cre⁻ mice. (Right axis and squares) The heart rate of each mouse was recorded during the course of the MAP measurement and the mean measurement is shown as black squares +/− standard deviation. Mean heart rates were not significantly different in the conditional VEGF knockouts compared with controls. Flt1-Cre⁺; VEGF^{(loxP/loxP)}, n=5; Flt1-Cre⁻, n=10; **p value<0.005.

FIG. 2, Panels a-c, illustrate that Flt1-Cre Transgene and VEGF-A are Co-expressed in Mesangial Cells of the Kidney Glomerulus: (a) (Left) H&E stained bright field images of sections from kidneys of mice aged 7.5 weeks subjected to in situ hybridization using a VEGF-A anti-sense probe. (Right) Dark field photographs of the images shown in the left panels. VEGF-A expression is markedly reduced within the kidney glomeruli of age-matched Flt1-Cre⁺; VEGF^{(loxP/loxP)} mice. (b) Immunohistochemical staining on kidney sections of embryonic Flt1-Cre⁺; VEGF^{(loxP/+)} mice aged 18.5 days using an affinity-purified antiserum raised against Cre-recombinase. (Top) Cre-recombinase expression (brown), driven by the Flt1 gene promotor, is detected in both endothelial and mesangial cells (me) within the glomerulus. (Bottom) Flt-Cre expression is shown in endothelial cells (en) throughout the tubulo-interstitial/medullary compartment as a control. (c) Real time RT-PCR analysis of total kidney RNA isolated from Flt1-Cre; VEGF-loxP mice aged 7 weeks. Relative RNA units (RRU) for Cre recombinase, Flt1, Flk1, and VEGF-A were normalized GAPDH levels and calculated from standard curves (Gerber et al., 2000). Flt1-Cre⁺; VEGF^{(loxP/loxP)}, n=6; Flt1-Cre⁺; VEGF^{(loxP/+)}, n=6; Flt1-Cre⁻, n=6; *p value<0.05.

FIG. 3, Panels a-m, schematically illustrate histologic Analysis of the Kidneys of Flt1-Cre; VEGF-LoxP Mice Aged 2 to 7 Weeks and Transmission Electron Micrographs of Kidneys Isolated from 5 Week Old Flt1-Cre; VEGF-LoxP Mice: (a) Kidney cortex of a Flt1-Cre⁺; VEGF^{(loxP/loxP)} mouse aged 2 weeks stained with H&E and photographed at low magnification. The boundary of a cortical cyst is marked (dotted line), and glomeruli, consisting of peripheral cells and apparently lacking glomerular capillary loops, are evident. (b) H&E stained section of a WT kidney glomerulus at 2 weeks of age. (c) H&E stained section of kidney glomeruli in a Flt1-Cre⁺; VEGF^{(loxP/loxP)} mouse aged 2 weeks. Tight capillary loops as observed in its WT littermate in (b), appear to be absent. (d) H&E stained section of a kidney glomerulus in a-Flt1-Cre⁺; VEGF^{(loxP/loxP)} mouse aged 2 weeks that is enlarged with glomerular capillaries that are obscured by abundant ECM deposition. (e) Typical appearance of a glomerulus from a Flt1-Cre⁻ mouse aged 7 weeks stained with H&E. (f) H&E stained section of a Flt1-Cre⁺; VEGF^{(loxP/loxP)} glomerulus aged 7 weeks. At this age, the glomeruli of the conditional VEGF knockouts are markedly enlarged and abundant ECM deposition impinges on the glomerular capillaries and urinary space. (g and h) Immunohistochemical staining of endothelial cells in Flt1-Cre⁻ (g) and Flt1-Cre⁺; VEGF^{(loxP/loxP)} (h) kidneys aged 7 weeks using α-CD31. Fewer CD31-positive endothelial cells are detected in the mutant kidneys. (i and j) TGF-β expression in Flt1-Cre⁻ (i) and Flt1-Cre⁺; VEGF^{(loxP/loxP)} (j) kidneys aged 7 weeks detected by in situ hybridization. Marked upregulation of TGF-β is detected in the conditional VEGF-A knockout kidneys. (k and l) Immunohistochemical staining to detect alpha smooth muscle actin (α-SMA) in kidneys of Flt1-Cre⁻ (k) and Flt1-Cre⁺; VEGF^{(loxP/loxP)} (l) mice aged 7 weeks. Increased α-SMA staining is detectable throughout the glomeruli of the conditional VEGF knockout, reflecting activation of the remaining mesangial cells. (m) (Top) Transmission electron micrographs demonstrate numerous defects in Flt1-Cre⁺; VEGF^{(loxP/loxP)} kidneys compared with (bottom) Flt1-Cre⁻ kidneys. (Left) There are areas of podocyte foot process (fp) fusion (arrows) in the diseased kidneys whereas there are distinct foot processes in controls. (Middle) In the mesangium (me), numerous electron-dense deposits (de) that are not present in the Flt-Cre⁻ kidney, can be seen in a subendothelial location consistent with immune-mediated glomerulonephritis. (Right) In glomeruli with advanced lesions, a markedly thickened and crinkled glomerular basement membrane is seen in the Flt1-Cre⁺; VEGF^{(loxP/loxP)} kidneys. Fenestrated endothelial cells (en) are seen in controls but are missing in the conditional VEGF knockouts.

FIG. 4, Panels a-b, schematically illustrate progression of kidney failure in Flt1-Cre⁺; VEGF^{(loxP/loxP)} mice is associated with IgM deposition and complement activation: (a) Fold change in the RNA levels of genes expressed on cells of the monocyte/macrophage (MAC-1, F4/80), B-cell (CD45R) and T-cell (Thy-1) lineages, in Flt1-Cre⁺; VEGF^{(loxP/loxP)} compared with Flt1-Cre⁻ kidney, lung, and heart tissue (black bars), and in Flt1-Cre⁺; VEGF^{(loxP/+)} compared with Flt1-Cre⁻ matched organs (grey bars). Expression levels have been standardized to the probe/primer sets specific for murine GAPDH. Statistically significant fold changes in expression are noted by asterisks, p value<0.005. Flt1-Cre⁺; VEGF^{(loxP/loxP)}, n=6; Flt1-Cre⁺; VEGF^{(loxP/+)}, n=6; Flt1-Cre⁻, n=6. (b) (Left) Immunohistochemical/fluorescent staining for cells of the monocyte/macrophage and (right) T-cell lineages in the kidneys of Flt1-Cre⁻ and Flt1-Cre⁺, VEGF^{(loxP/loxP)} mice aged 5 weeks, using α-F4/80 and α-CD4 antibody respectively. Monocyte/macrophages and a subset of T-cells are recruited into the kidney tissue of the conditional VEGF-A knockouts.

FIG. 5, Panels a-f, illustrates In Vitro Analysis of VEGF-A and Flt1-Deficient Mesangial Cells: (a) Gene-targeting strategy to create Flt1-loxP mice. The targeting vector was designed to introduce a PGK-Neo cassette flanked by 2 loxP sites (LoxP1 and LoxP2) upstream of the first exon containing the translation initiation codon (ATG) of the Flt1 gene, and to introduce a third loxP site (LoxP3) 3′ to the first exon. Following Cre-recombinase expression, embryonic stem (ES) cell clones that had undergone recombination between LoxP1 and LoxP2 were selected and used to generate Flt1-loxP mice. The positions of the PCR-amplified genomic DNA probes (5′ Pr, 3′ Pr) used to screen for targeting events and recombination by Southern blotting are shown. The position of restriction enzyme sites used in this screening and the size of the regions of the targeting vector (in kilobases) are as indicated. E: EcoRI; H: HindIII; K: KpnI; kb: kilobases. (b) Southern blot analysis of genomic DNA extracted from WT and targeted (Targ.) ES cell clones, and from Flt1^{(loxP/loxP)} mesangial cells (Mes.) infected with adenovirus encoding LacZ (LZ) or Cre-recombinase (Cre) genes. Genomic DNA from the respective cells was digested with either EcoRI or both HindIII and KpnI to analyse for targeting events and loxP recombination at either the 5′ end or 3′ end of the targeted regions of the Flt1 gene respectively. The sizes of the expected fragments detected in each of the lanes is indicated (in kilobases) to the left and right of each panel respectively. (c) VEGF-A expression in mesangial cells infected with adenovirus. Mesangial cells were isolated from WT and VEGF^{(loxP/loxP)} mice and infected with adenovirus encoding LacZ (Ad-LacZ) or Cre-recombinase (Ad-Cre). Total RNA was isolated and subjected to quantitative real-time PCR for the analysis of VEGF-A expression. Results are expressed as relative RNA units (RRU) following standardization to GAPDH, and standard curves for each primer/probe set were generated using total kidney RNA from WT mice. (d) Flt1 expression in mesangial cells infected with adenovirus. Mesangial cells from WT and Flt1^{(loxP/loxP)} mice were isolated and infected with Ad-LacZ and Ad-Cre. RNA was isolated and analysed for Flt1 expression by quantitative RT-PCR. Results are expressed as described in C. (e) Survival of VEGF-A and Flt1-deficient mesangial cells in vitro. The cell count ratio between Ad-LacZ and Ad-Cre treatments was calculated and normalized as a percentage of the value obtained for the WT cells. Both VEGF-A and Flt1-deficient mesangial cells exhibited significantly reduced survival in vitro compared with WT mesangial cells. Statistically significant differences in survival compared to WT cells are noted by asterisks: *p value<0.05; **p value<0.01. Flt1-deficient and VEGF-deficient mesangial cells also exhibit significantly different survival in vitro, p<0.05. (f) Survival of Ad-LacZ infected mesangial cells cultured in the presence of a neutralizing VEGF antibody (α-VEGF) or control antibody (control IgG). The decrease in mesangial cell survival evident when either VEGF-A or Flt1 is ablated genetically was not recapitulated by culturing mesangial cells in the presence of α-VEGF.

FIG. 6, Panels a-d, illustrate real time RT-PCR analysis of total kidney RNA isolated from 7 week old Flt1-Cre; VEGF-loxP mice to detect expression of different forms of collagen. Relative RNA units (RRU) for collagen α1 type I (a), collagen α2 type II (b), collagen α2 type IV (c) and collagen al type XVIII (d) were normalized to glyceraldehydes-3-dehydrogenase (GAPDH) levels and calculated from standard curves.

### DETAILED DESCRIPTION

### Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

Nephritis is an inflammation of the kidneys. Evidence, e.g., blood and/or protein in the urine and impaired kidney function, etc., of nephritis depends on the type, location, and intensity of the immune response, inflammation affecting the glomeruli, the tubules, the tissue around the tubules, or blood vessels. “Nephritis-related disease” include, but are not limited to, e.g., primary glomerulopathies (acute diffuse proliferative glomerulonephritis, post-streptococcal glomerulopathy, non-post streptococcal glomerulopathy, crescentic glomerulonephritis, membraneous glomerulopathy, lipoid nephrosis, focal segmental glomerulosclerosis, membranoproliferative glomerulonephritis, IgA nephropathy, focal proliferative glomerulonephritis, and chronic glomerulonephritis), systemic diseases (systemic lupus erythematosus, diabetes mellitus, amyloidosis, Goodpasture's syndrome, polyarteritis nodosa, Welgener's granulomatosis, Henoch-Schonlein purpura, and Bacterial endocarditis), and hereditary disorders (Alport's syndrome, thin membrane disease, and Fabry's disease).

“Nephrotic syndrome” is a collection of symptoms caused by many diseases that affect the kidneys, resulting in a severe, prolonged loss of protein into the urine, decreased blood levels of protein (especially albumin), retention of excess salt and water in the body, and increased levels of fats (lipids) in the blood. Nephrotic syndrome can be caused by any of the glomerulopathies or a vast array of diseases. Typically, the syndrome progresses to complete kidney failure in 3 or 4 months.

“Acute nephritic syndrome” or “acute glomerulonephritis” refers to an inflammation of the glomeruli that often results in the sudden appearance of blood in the urine, with clumps of red blood cells (casts) and variable amounts of protein in the urine. Acute nephritic syndrome may follow a streptococcal infection, such as strep throat. The glomeruli are damaged by the accumulation of antigen from the dead streptococci clumped together with the antibodies that neutralize them. These clumps (immune complexes) coat the membranes of the glomeruli and interfere with their filtering function. Acute nephritic syndrome may also be caused by a reaction to other infections, such as infection of an artificial body part (prosthesis), bacterial endocarditis, pneumonia, abscesses of abdominal organs, chickenpox, infectious hepatitis, syphilis, and malaria. The last three infections may cause nephrotic syndrome rather than acute nephritic syndrome. “Chronic nephritic syndrome” or “chronic glomerulonephritis” refers to a disorder occurring in several diseases in which the glomeruli are damaged and kidney function degenerates over a period of years.

Glomerulopathy is a glomerular disease, which is a disease of a plexus of capillaries. In kidney glomerular disease, it is a disease of the tuft formed of capillary loops at the start of each nephric tubule. Types of glomerulopathies, include, but are not limited to, e.g., (1) Acute nephritic syndrome; (2) Rapidly progressive nephritic syndrome; (3) Nephrotic syndrome; and (4) Chronic nephritic syndrome. Because the glomerulus is damaged, substances not normally filtered out of the bloodstream, such as proteins, blood, white blood cells, and debris, can pass through the glomerulus and enter the urine. Tiny blood clots (microthrombi) may form in the capillaries that supply the glomerulus.

Glomerulosclerosis is a degenerative kidney disease that results in hyaline deposits or scarring within the renal glomeruli often associated with renal arteriosclerosis or diabetes. Typically, there is an infiltration of circulating inflammatory cells, fibrosis of interstitium and tubular atrophy. Glomerular injury caused by several factors brings about proteinuria in which proteins bind with soluble immunoglobulin A (sIgA), sIgG and sIgM, forming immune complexes on the basement membrane. These immune complexes function as a chemotactic factor for inflammatory lymphocytes, which cause excessive immune responses in the affected areas (Bohle A et al., Kidney Int 67 (Suppl.):186S-188S(1998)). When tubules are damaged by inflammatory cells, blood vessels connected with glomeruli are also injured and occluded. As a consequence, glomeruli become adversely affected and deteriorate. These glomerular changes are accompanied by tissue fibrosis and progress into eventual renal failure (see, e.g., Ratscchek M et al., Clin Nephrol 25: 221-226 (1986); Bohle A et al, Clin Nephrol 29: 28-34 (1998); Bohle A et al., Kidney Blood Press Res 19:191-195 (1996)).

One type is Focal Segmental Glomerulosclerosis (FSGS) with is a segmental collapse of the glomerular capillaries with thickened basement membranes and increased mesangial matrix, which often results in proteinuria and renal insufficiency. See, e.g., Kamanna et al., Histol. Histopathol. 13: 169-179 (1998); Wehrmann et al., Clin. Nephrol. 33:115-122 (1990); Mackensen-Haen, et al., Clin. Nephrol. 37:70-77 (1992). It can cause permanent kidney failure.

The term “mesangium” refers to a tissue supporting capillary loops in the glomerulus of the kidney and composed of mesangial cells and mesangial matrix. Mesangial cells are known to maintain the loop structure of the glomerulus as well as have a phagocytic function or the ability to regulate glomerular blood flow. Mesangial cells have angiotensin II receptors and produce platelet-activating factor, prostaglandin, type IV collagen, fibronectin, etc. The mesangial matrix is an extracellular matrix component that surrounds mesangial cells.

The term “VEGF receptor” or “VEGFR” as used herein refers to a cellular receptor for VEGF, ordinarily a cell-surface receptor found on vascular endothelial cells, as well as fragments and variants thereof which retain the ability to bind VEGF (such as fragments or truncated forms of the extracellular domain). Some examples of VEGFR include the protein kinase receptors referred to in the literature as Flt-1 (also used interchangeably herein “VEGFR-1”) and KDR/Flk-1 (also used interchangeably herein “VEGFR-2”). See, e.g., DeVries et al. Science, 255:989 (1992); Shibuya et al. Oncogene, 5:519 (1990); Matthews et al. Proc. Nat. Acad. Sci., 88:9026 (1991); Terman et al. Oncogene, 6:1677 (1991); and Terman et al. Biochem. Biophys. Res. Commun., 187:1579 (1992). The Flt-1 (fms-like-tyrosine kinase) and KDR (kinase domain region) receptors bind VEGF with high affinity. Flk-1 (fetal liver kinase-1), the murine homolog of KDR, shares 85% sequence identity with human KDR. Ferrara Kidney Intl. 56:794-814 (1999). Both Flt-1 and KDR/Flk-1 have seven immunoglobulin (Ig)-like domains in the extracellular domain (ECD), a single transmembrane region and a consensus tyrosine kinase (TK) sequence, which is interrupted by a kinase-insert domain. Flt-1 has the highest affinity for rhVEGF₁₆₅, with a Kd of approximately 10 to 20 pM. KDR has a lower affinity for VEGF, with a Kd of approximately 75 to 125 pM. The nucleic acid sequences and amino acids sequences of a VEGFR are readily accessible and obtainable by one of skill in the art.

Other VEGF receptors include those that can be cross-link labeled with VEGF, or that can be co-immunoprecipitated with KDR or Flt-1. An additional VEGF receptor that binds VEGF₁₆₅ but not VEGF₁₂₁ has been identified, which is neuropilin 1. Soker et al Cell 92:735-45 (1998). The isoform-specific VEGF binding receptor is also a receptor for the collapsin/semaphorin family that mediates neuronal cell guidance.

The Flt-1 and KDR receptors mainly exist as a bound receptor on the surface of vascular endothelial cells, although they can also be present in non-endothelial cells. Some soluble forms of VEGFR have also been found. For example, a cDNA coding an alternatively spliced soluble form of Flt-1 (sFlt-1), lacking the seventh Ig-like domain, transmembrane sequence, and the cytoplasmic domain, has been identified in human umbilical vein endothelial cells (HUVECs). Kendall et al. Biochem. Biophys. Res. Comm. 226:324-328 (1996)

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol. Endocrin., 5:1806 (1991), and, Robinson & Stringer, Journal of Cell Science, 144(5):853-865 (2001), together with the naturally occurring allelic and processed forms thereof. The term “VEGF” is also used to refer to fragments of the polypeptide, e.g., comprising amino acids 8 to 109 or 1 to 109 of the 165-amino acid human vascular endothelial cell growth factor, that retain biological activity. Reference to any such forms of VEGF may be identified in the application, e.g., by “VEGF (8-109),” “VEGF (1-109)” or “VEGF165.” The amino acid positions for a “fragment” native VEGF are numbered as indicated in the native VEGF sequence. For example, amino acid position 17 (methionine) in fragment native VEGF is also position 17 (methionine) in native VEGF. The fragment native VEGF can have binding affinity for the KDR and/or Flt-1 receptors comparable to native VEGF.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family (A, B, C, D, and E), PlGF, PDGF family, fibroblast growth factor family (FGFs), TIE ligands (Angiopoietins), ANGPTL3, ANGPTL4, ephrins, etc. It would also include factors that accelerate wound healing, such as growth hormone, insulin-like growth factor-I (IGF-I), VIGF, epidermal growth factor (EGF), CTGF and members of its family, and TGF-α and TGF-β. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003) (e.g., Table 1 listing known angiogenic factors); and, Sato Int. J. Clin. Oncol., 8:200-206 (2003).

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

A polypeptide “variant” means a biologically active polypeptide having at least about 80% amino acid sequence identity with the corresponding native sequence polypeptide, or fragment thereof. Such variants include, for instance, polypeptides wherein one or more amino acid residues are added, or deleted, at the N- and/or C-terminus of the polypeptide. Ordinarily, a variant will have at least about 80% amino acid sequence identity, or at least about 90% amino acid sequence identity, or at least about 95% or more amino acid sequence identity with the native sequence polypeptide, or fragment thereof.

The term “variant” as used herein refers to a protein variant as described herein and/or which includes one or more amino acid mutations in the native protein sequence. Optionally, the one or more amino acid mutations include amino acid substitution(s). Variants thereof for use in the invention can be prepared by a variety of methods well known in the art. In certain embodiments of the invention, the VEGF employed in the methods of the invention comprises recombinant VEGF₁₆₅. Amino acid sequence variants can be prepared by mutations in the, e.g., VEGF DNA or VEGFR DNA. Such variants include, for example, deletions from, insertions into or substitutions of residues within the amino acid sequence of VEGF or VEGFR. Any combination of deletion, insertion, and substitution may be made to arrive at the final construct having the desired activity. The mutations that will be made in the DNA encoding the variant must not place the sequence out of reading frame and preferably will not create complementary regions that could produce secondary mRNA structure. EP 75,444A.

Variants optionally are prepared by site-directed mutagenesis of nucleotides in the DNA encoding the native protein or phage display techniques, thereby producing DNA encoding the variant, and thereafter expressing the DNA in recombinant cell culture.

While the site for introducing an amino acid sequence variation is predetermined, the mutation per se need not be predetermined. For example, to optimize the performance of a mutation at a given site, random mutagenesis may be conducted at the target codon or region and the expressed variants screened for the optimal combination of desired activity. Techniques for making substitution mutations at predetermined sites in DNA having a known sequence are well-known, such as, for example, site-specific mutagenesis. Preparation of the variants described herein can be achieved by phage display techniques, such as those described in the PCT publication WO 00/63380.

After such a clone is selected, the mutated protein region may be removed and placed in an appropriate vector for protein production, generally an expression vector of the type that may be employed for transformation of an appropriate host.

Amino acid sequence deletions generally range from about 1 to 30 residues, optionally 1 to 10 residues, optionally 1 to 5 or less, and typically are contiguous.

Amino acid sequence insertions include amino- and/or carboxyl-terminal fusions of from one residue to polypeptides of essentially unrestricted length as well as intrasequence insertions of single or multiple amino acid residues. Intrasequence insertions (i.e., insertions within the native protein sequence) may range generally from about 1 to 10 residues, optionally 1 to 5, or optionally 1 to 3. An example of a terminal insertion includes a fusion of a signal sequence, whether heterologous or homologous to the host cell, to the N-terminus to facilitate the secretion from recombinant hosts.

Additional variants are those in which at least one amino acid residue in the native protein has been removed and a different residue inserted in its place. Such substitutions may be made in accordance with those shown in Table 1. Variants can also comprise unnatural amino acids as described herein.

Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

“Naturally occurring amino acid residues” (i.e. amino acid residues encoded by the genetic code) may be selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include, e.g., norleucine, ornithine, norvaline, homoserine and other amino acid residue analogues such as those described in Ellman et al. Meth. Enzym. 202:301-336 (1991) & us Patent application publications 20030108885 and 20030082575. Briefly, these procedures involve activating a suppressor tRNA with a non-naturally occurring amino acid residue followed by in vitro or in vivo transcription and translation of the RNA. See, e.g., US Patent application publications 20030108885 and 20030082575; Noren et al. Science 244:182 (1989); and, Ellman et al., supra.

The effect of the substitution, deletion, or insertion may be evaluated readily by one skilled in the art using routine screening assays. For example, a phage display-selected VEGF variant may be expressed in recombinant cell culture, and, optionally, purified from the cell culture. The VEGF variant may then be evaluated for KDR or Flt-1 receptor binding affinity and other biological activities, such as those known in the art or disclosed in the application. The binding properties or activities of the cell lysate or purified VEGF variant can be screened in a suitable screening assay for a desirable characteristic. For example, a change in the immunological character of the VEGF variant as compared to native VEGF, such as affinity for a given antibody, may be desirable. Such a change may be measured by a competitive-type immunoassay, which can be conducted in accordance with techniques known in the art. The respective receptor binding affinity of the VEGF variant may be determined by ELISA, RIA, and/or BLAcore assays, known in the art and described further in the Examples below. In one embodiment of the invention, VEGF variants of the invention will also exhibit activity in KIRA assays reflective of the capability to induce phosphorylation of the KDR receptor. In one embodiment of the invention, VEGF variants of the invention will additionally or alternatively induce endothelial cell proliferation (which can be determined by known art methods such as the HUVEC proliferation assay). In addition to the specific VEGF variants disclosed herein, the VEGF variants described in Keyt et al. J. Biol. Chem. 271:5638-5646 (1996) are also contemplated for use in the invention.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, e.g., digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “modulates” or “modulation” as used herein refers to the decrease, inhibition, reduction, amelioration, increase or enhancement of a VEGFR gene function, expression, activity, or alternatively a phenotype associated with VEGFR gene.

The term “VEGFR modulator” or “VEGFR modulating agent” or “VEGFR modulating compound” refers to a molecule that can activate, e.g., an agonist, its expression, or that can inhibit, e.g., an antagonist (or inhibitor), the activity of a VEGFR or its expression. The term “agonist” is used to refer to an agent that has the ability to signal through a native VEGFR receptor; The term “agonist” is defined in the context of the biological role of a VEGFR receptor. In certain embodiments of the invention, a VEGFR modulator includes, but is not limited to, a VEGFR agonist, e.g., a Flt1 agonist, a ligand that binds to a VEGFR receptor, e.g., VEGF, VEGF selective variants, PlGF, VEGF-B, VEGF-C, and VEGF-D. Additonal agonists of the invention include but are not limited to, e.g., VEGFR variants with agonist activity, VEGFR agonist antibodies, etc.

An VEGFR antagonist refers to a molecule capable of neutralizing, blocking, inhibiting, abrogating, reducing or interfering with VEGFR activities, e.g., cell proliferation or growth, migration, adhesion or metabolic, including its binding to ligand, e.g., VEGF, VEGF selective variants, PlGF and VEGF-B, VEGF-C, and VEGF-D. VEGFR antagonists include, e.g., anti-VEGFR antibodies and antigen-binding fragments thereof, receptor molecules and derivatives which bind specifically to VEGFR thereby sequestering its binding to one or more ligands, anti-VEGFR antibodies and VEGFR antagonists such as small molecule inhibitors of the receptor. Other VEGFR antagonists also include antagonist variants of VEGFR, antisense molecules (e.g., VEGFR-SiRNA), RNA aptamers, and ribozymes against VEGFR or its receptor. In certain embodiments, antagonist VEGFR antibodies are antibodies that inhibit or reduce the activity of VEGFR by binding to a specific subsequence or region of VEGFR.

The term “Anti-VEGFR antibody” is an antibody that binds to VEGFR with sufficient affinity and specificity. In certain embodiments of the invention, the anti-VEGFR antibody of the invention can be used as a therapeutic agent in targeting and interfering with diseases or conditions wherein VEGFR activity is involved. Generally, an anti-VEGFR antibody will usually not bind to other VEGFR homologues.

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments (see below) so long as they exhibit the desired biological activity.

Unless indicated otherwise, the expression “multivalent antibody” is used throughout this specification to denote an antibody comprising three or more antigen binding sites. The multivalent antibody is typically engineered to have the three or more antigen binding sites and is generally not a native sequence IgM or IgA antibody.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked-by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) or Marks et al., J. Mol. Biol. 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunuoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., BioTechnology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG₁ (including non-A and A allotypes), IgG₂, IgG₃, IgG₄, IgA, and IgA₂. The heavy-chain constant domains that correspond to the different classes of antibodies are called α, δ, ε, γ and μ, respectively. The subunit structures and three-dimensional configurations of different classes of immunoglobulins are well known.

The light chains of antibodies from any vertebrate species can be assigned to one of two clearly distinct types, called kappa (6) and lambda (8), based on the amino acid sequences of their constant domains.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The Fc region of an immunoglobulin generally comprises two constant domains, a CH2 domain and a CH3 domain, and optionally comprises a CH4 domain. By “Fc region chain” herein is meant one of the two polypeptide chains of an Fc region.

The “CH2 domain” of a human IgG Fc region (also referred to as “Cg2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333, expressly incorporated herein by reference). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions. In certain embodiments of the invention, an antibody of the invention may have an altered Fc region resulting in altered effector function, e.g., enhanced function or reduced function.

A “native sequence Fc region” comprises an amino acid sequence identical to the amino acid sequence of an Fc region found in nature.

A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will typically possess, e.g., at least about 80% sequence identity with a native sequence Fc region and/or with an Fc region of a parent polypeptide, or at least about 90% sequence identity therewith, or at least about 95% sequence or more identity therewith.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Typically, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being generally preferred. The effector cells may be isolated from a native source thereof, e.g. from blood or PBMCs as described herein.

The terms “Fc receptor” and “FcR” are used to describe a receptor that binds to the Fc region of an antibody. In one embodiment, FcR is a native sequence human FcR. In one embodiment, FcR is one which binds an IgG antibody (a gamma receptor) and includes receptors of the FcγRI, FcγRII, and FcγRIII subclasses, including allelic variants and Immunol. Today alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRN, which is responsible for the transfer of maternal IgGs to the fetus (Guyer et al., J. Immunol. 117:587 (1976) and Kim et al., J. Immunol. 24:249 (1994) and the regulation of homeostasis of immunoglobulins. Methods of measuring binding to FcRn are known (see, e.g., Ghetie and Ward, Immunol Today 18(12):592-598 (1997); Ghetie et al., Nature Biotechnology, 15(7):637-640 (1997); Hinton et al., J. Biol. Chem. 279(8):6213-6216 (2004); and WO2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO2000/42072 describes antibody variants with improved or diminished binding to FcRs. See also, e.g., Shields et al., J. Biol. Chem. 9(2): 6591-6604 (2001).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

An antibody having a “biological characteristic” of a designated antibody is one which possesses one or more of the biological characteristics of that antibody which distinguish it from other antibodies that bind to the same antigen. In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

A “polypeptide chain” is a polypeptide wherein each of the domains thereof is joined to other domain(s) by peptide bond(s), as opposed to non-covalent interactions or disulfide bonds.

A “flexible linker” herein refers to a peptide comprising two or more amino acid residues joined by peptide bond(s), and provides more rotational freedom for two polypeptides (such as two Fd regions) linked thereby. Such rotational freedom allows two or more antigen binding sites joined by the flexible linker to each access target antigen(s) more efficiently. Examples of suitable flexible linker peptide sequences include gly-ser, gly-ser-gly-ser, ala-ser, and gly-gly-gly-ser.

A “dimerization domain” is formed by the association of at least two amino acid residues (generally cysteine residues) or of at least two peptides or polypeptides (which may have the same, or different, amino acid sequences). The peptides or polypeptides may interact with each other through covalent and/or non-covalent association(s). Examples of dimerization domains herein include an Fc region; a hinge region; a CH3 domain; a CH4 domain; a CH1-CL pair; an “interface” with an engineered “knob” and/or “protruberance” as described in U.S. Pat. No. 5,821,333, expressly incorporated herein by reference; a leucine zipper (e.g. a jun/fos leucine zipper, see Kostelney et al., J. Immunol., 148: 1547-1553 (1992); or a yeast GCN4 leucine zipper); an isoleucine zipper; a receptor dimer pair (e.g., interleukin-8 receptor (IL-8R); and integrin heterodimers such as LFA-1 and GPIIIb/IIIa), or the dimerization region(s) thereof; dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF); see Arakawa et al. J. Biol. Chem. 269(45): 27833-27839 (1994) and Radziejewski et al. Biochem. 32(48): 1350 (1993)), or the dimerization region(s) thereof; a pair of cysteine residues able to form a disulfide bond; a pair of peptides or polypeptides, each comprising at least one cysteine residue (e.g. from about one, two or three to about ten cysteine residues) such that disulfide bond(s) can form between the peptides or polypeptides (hereinafter “a synthetic hinge”); and antibody variable domains. The most preferred dimerization domain herein is an Fc region or a hinge region.

The phrase “stimulating proliferation of a cell” encompasses the step of increasing the extent of growth and/or reproduction of the cell relative to an untreated cell or a reduced treated cell either in vitro or in vivo. An increase in cell proliferation in cell culture can be detected by counting the number of cells before and after exposure to a molecule of interest. The extent of proliferation can be quantified via microscopic examination of the degree of confluence. Cell proliferation can also be quantified using assays known in the art, e.g., thymidine incorporation assay, and commercially available assays. The phrase “inhibiting proliferation of a cell” encompasses the step of decreasing the extent of growth and/or reproduction of the cell relative to an untreated cell or a reduced treated cell either in vitro or in vivo. It can be quantified as described above.

“Subject” for purposes of treatment refers to any animal. Generally, the animal is a mammal. “Mammal” for purposes of treatment refers to any animal classified as a mammal, including humans, domestic and farm animals, and zoo, sports, or pet animals, such as dogs, horses, cats, cows, sheep, pigs, etc. Typically, the mammal is a human.

Administration “in combination with” one or more further therapeutic agents includes simultaneous (concurrent) and/or consecutive administration in any order.

The term “effective amount” or “therapeutically effective amount” refers to an amount of a drug effective to treat a disease or disorder in a subject. In the case of kidney disease, the effective amount of the drug may reduce the symptoms or lessen or eliminate the disease.

“Treatment” refers to both therapeutic treatment and prophylactic or preventative measures. Those in need of treatment include those already with the disorder as well as those in which the disorder is to be prevented.

A “disorder” is any condition that would benefit from treatment with a molecule of the invention, e.g., see the kidney disorders described herein. This includes chronic and acute disorders or diseases including those pathological conditions which predispose the subject to the disorder in question.

“Transfection” refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

“Transformation” refers to introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described by Cohen, Proc. Natl. Acad. Sci. (USA), 69: 2110 (1972); and, Mandel et al. J. Mol. Biol., 53: 154 (1970), is generally used for prokaryotes or other cells that contain substantial cell-wall barriers. For mammalian cells without such cell walls, the calcium phosphate precipitation method of Graham and van der Eb, Virology, 52: 456-457 (1978), is often used. General aspects of mammalian cell host system transformations have been described by Axel in U.S. Pat. No. 4,399,216 issued Aug. 16, 1983. Transformations into yeast are typically carried out according to the method of Van Solingen et al. J. Bact., 130: 946 (1977) and Hsiao et al. Proc. Natl. Acad. Sci. (USA), 76: 3829 (1979). However, other methods for introducing DNA into cells such as by nuclear injection or by protoplast fusion may also be used.

### Kidney Disease

A critical determinant of glomerular matrix accumulation is the balance between ECM synthesis and dissolution (see, e.g., Schnaper, H. W. (1995). Balance between matrix synthesis and degradation: a determinant of glomerulosclerosis. Pediatr Nephrol 9, 104-111). When this balance is disrupted a kidney disorder develops. For example, glomerulosclerosis is a process by which normal, functional glomerular tissue is replaced by accumulated deposits of extracellular matrix (ECM). Long term exposure of current treatments for glomerulosclerosis (e.g., steroids, cyclosporine, etc.) often induces side effects to several organs. In addition, the current treatments do not necessarily interrupt or reverse progression of the disease thus still requiring further treatments such as kidney dialysis or kidney transplant.

Renal diseases with down-regulation of VEGF frequently correlate with glomerulosclerosis and auto-immune deposits. See, e.g., Shulman, K., et al. Expression of vascular permeability factor (VPF/VEGF) is altered in many glomerular diseases. J Am Soc Nephrol 7:661-666 (1996a); Noguchi, K., et al. Activated mesangial cells produce vascular permeability factor in early-stage mesangial proliferative glomerulonephritis. J Am Soc Nephrol 9:1815-1825 (1998); Shulman, K., et al. Expression of vascular permeability factor (VPF/VEGF) is altered in many glomerular diseases. J-Am-Soc-Nephrol 7:661-666 issn: 1046-6673 (1996b); Wada, Y., et al. (2002). Impairment of vascular regeneration precedes progressive glomerulosclerosis in anti-Thy 1 glomerulonephritis. Kidney Int 61:432-443); and, Yuan, H. T., et al. (2002). Angiopoietin correlates with glomerular capillary loss in anti-glomerular basement membrane alomerulonephritis. Kidney Int 61:2078-2089).

The application describes a function of VEGF-A and VEGFR in kidney cells during kidney development. Interference with such function induces glomerulosclerosis in mice. The invention provides methods for treating a pathological kidney condition in a subject with a modulator of VEGFR. The phrase “pathological kidney condition” is used interchangeably with “kidney disorder” or “kidney disease” or “renal disease” to indicate any structural and/or functional kidney abnormalities. A modulator of VEGFR includes, but is not limited to, a VEGFR ligand, e.g., VEGF (A, B, C, D and/or E), a Flt-1 agonist (e.g., a Flt-selective VEGF variant, VEGF-B, PlGF), VEGFR agonist antibodies, VEGFR agonist small molecules, etc., which can be a therapeutic for treating kidney disease, e.g., inflammatory kidney disease, glomerulosclerosis, etc. In certain embodiments, the kidney disease is caused by an infection. In certain embodiments, the subject is being treated for the kidney disease with other agents, e.g., steroids, cyclosporine, etc.). In certain embodiments of the invention, an effective amount of a Flt1 agonist is administered to a subject in order to treat the pathological kidney condition. In one embodiment of the invention, a KDR agonist or other angiogenic factor can be administered in combination with a Flt1 agonist, e.g., at a lower ratio than Flt1, which can result in a maximal therapeutic benefit, by providing stimulation of angiogenesis. In certain embodiments of the invention, a KDR agonist or other angiogenic factor can be administered in combination with a Flt1 agonist, e.g., at a higher ratio than Flt1 or an equal ratio. In another embodiment, a VEGF variant that preferentially activates Flt-1 versus KDR can be used to combine optimal characteristics of safety and efficacy. In certain embodiments, VEGF is administered in combination with a Flt1 agonist.

Treatment of the a pathological kidney condition can be assessed by those of skill in the art, e.g., by histological analysis and immunocytochemistry, by urine analysis, e.g., blood urea nitrogen (B.U.N), serum creatine, etc., by measuring of mean arterial blood pressure, etc. In certain embodiments of the invention, the inflammatory kidney disease is characterized by and treatment is assessed by alterations in inflammatory cells, immune complex depositions, e.g., IgM deposition. or complement activation in affected glomeruli, e.g., activation of C1q, C3 and C4. In certain embodiments of the invention, the renal disease comprises alterations in kidney mesangial cells, e.g., a decrease in VEGF levels, while treatment would have the opposite effect. In certain embodiments of the invention, glomerulonephritis is determined by and treatment is assessed by measuring proteinuria, glomerular sclerosis, hypertension, or a combination thereof. It also can be assessed by determining survival of kidney cells, e.g., kidney mesangial cells, gene expression of ECM synthesis or matrix degradation. In such cases, the glomerulonephritis can be determined by decreased survival of kidney mesangial cells, an increase in gene expression of ECM synthesis, a reduction in matrix degradation or a combination thereof, while treatment would have the opposite effects.

Compositions of the Invention and their Production

The invention relates to uses of various agents capable of modulating VEGFR, e.g., VEGFR-1 and VEGFR-2, activities in the kidney. The term “agent” or, alternatively, “compound” as used herein refers broadly to any substance with identifiable molecular structure and physiochemical property. Non-limiting examples of agents capable of modulating VEGFR activities include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like.

The VEGFR modulating agents encompassed by the invention can be either an agonist of a VEGFR. For example, a VEGFR agonist can be a growth factor ligand (e.g., VEGF, VEGF B, VEGF C, VEGF D, VEGF E, PlGF, etc. (typically, VEGF, VEGF B and/or PlGF)) or an antibody that binds to the VEGFR's extracellular domain and triggers its signal transduction activity. Alternatively, a VEGFR agonist can be a small molecule compound that binds to the VEGFR's cytoplasmic domain and mediates its tyrosine phosphorylation.

In one embodiment, the agonist of the invention is “selective” or “specific” to Flt-1, i.e., it exclusively or preferably modulates Flt-1 over other receptor tyrosine kinases such as KDR. In another embodiment, the agonist of the invention is “selective” or “specific” to KDR, i.e., it exclusively or preferably modulates KDR over other receptor tyrosine kinases such as Flt-1. In one aspect, the VEGFR agonist of the invention comprises a VEGF variant polypeptide capable of selectively binding to Flt-1 (referred hereinafter as “Flt-1 selective VEGF variant”, or “Flt1-sel”, or “Flt1sel”). In one aspect, the VEGFR agonist is VEGF-B or PlGF, which selectively bind to Flt1.

Flt-sel and methods of making the same have been known and are described in the Example sections below. Additional disclosures relating to Flt-sel can be found in, for example, the PCT publication WO 00/63380 and Li et al. J. Biol. Chem. 275:29823-29828 (2000). In certain embodiments of the invention, Flt-sel variants include one or more amino acid mutations and exhibit binding affinity to the Flt-1 receptor which is equal to or greater (>) than the binding affinity of native VEGF to the Flt-1 receptor, and even more preferably, such VEGF variants exhibit less binding affinity (<) to the KDR receptor than the binding affinity exhibited by native VEGF to KDR. When binding affinity of such VEGF variant to the Flt-1 receptor is approximately equal (unchanged) or greater than (increased) as compared to native VEGF, and the binding affinity of the VEGF variant to the KDR receptor is less than or nearly eliminated as compared to native VEGF, the binding affinity of the VEGF variant, for purposes herein, is considered “selective” for the Flt-1 receptor. In one embodiment of the invention, a Flt-1 selective VEGF variants of the invention will have at least 10-fold less binding affinity to KDR receptor (as compared to native VEGF), and even more preferably, will have at least 100-fold less binding affinity to KDR receptor (as compared to native VEGF). The respective binding affinity of the VEGF variant may be determined by ELISA, RIA, and/or BIAcore assays, known in the art and described in the PCT publication WO 00/63380.

In some aspects of the invention, various methods for kidney treatment further comprise administering an agent capable of modulating KDR activities. For example, a KDR agonist can be administered in combination with a Flt-1 agonist to treat kidney disease. KDR has been identified as the major receptor tyrosine kinase that mediates VEGF's activities in endothelial cell proliferation.

In one aspect, the KDR agonist comprises VEGF (as well as VEGF-C or VEGF-D) or a VEGF variant polypeptide capable of selectively binding to KDR (referred hereinafter as “KDR selective VEGF variant”, or “KDR-sel”, or “KDRₛₑₗ”). KDR-sel VEGF variants and methods of making the same are described in detail in, for example, the PCT publication WO 00/63380 and Li et al. J. Biol. Chem. 275:29823-29828 (2000). In one embodiment, the KDR-sel include one or more amino acid mutations and exhibit binding affinity to the KDR receptor which is equal to or greater (>) than the binding affinity of native VEGF to the KDR receptor, and even more preferably, the VEGF variants exhibit less binding affinity (<) to the fit-1 receptor than the binding affinity exhibited by native VEGF to Flt-1. When binding affinity of such VEGF variant to the KDR receptor is approximately equal (unchanged) or greater than (increased) as compared to native VEGF, and the binding affinity of the VEGF variant to the flt-1 receptor is less than or nearly eliminated as compared to native VEGF, the binding affinity of the VEGF variant, for purposes herein, is considered “selective” for the KDR receptor. In one embodiment of the invention, a KDR-sel of the invention will have at least 10-fold less binding affinity to Flt-1 receptor (as compared to native VEGF), and even more preferably, will have at least 100-fold less binding affinity to Flt-1 receptor (as compared to native VEGF). The respective binding affinity of the VEGF variant may be determined by ELISA, RIA, and/or BIAcore assays that are known in the art. In one embodiment of the invention, a KDR-sel of the invention will also exhibit activity in KIRA assays reflective of the capability to induce phosphorylation of the KDR receptor. In one embodiment of the invention, KDR selective VEGF variants of the invention will additionally or alternatively induce endothelial cell proliferation (which can be determined by known methods such as the HUVEC proliferation assay).

In one aspect, the VEGFR modulating agents of the invention are produced by recombinant methods. Isolated DNA used in these methods is understood herein to mean chemically synthesized DNA, cDNA, chromosomal, or extrachromosomal DNA with or without the 3′- and/or 5′-flanking regions. In certain embodiments of the invention, VEGFR modulating agents are made by synthesis in recombinant cell culture.

For such synthesis, a nucleic acid that encodes a VEGF or VEGFR or variants thereof is needed. DNA encoding a VEGF molecule may be obtained from pituitary follicular cells, e.g., bovine pituitary follicular cells, by (a) preparing a cDNA library from these cells, (b) conducting hybridization analysis with labeled DNA encoding the VEGF or fragments thereof (up to or more than 100 base pairs in length) to detect clones in the library containing homologous sequences, and (c) analyzing the clones by restriction enzyme analysis and nucleic acid sequencing to identify full-length clones. If full-length clones are not present in a cDNA library, then appropriate fragments may be recovered from the various clones using the nucleic acid sequence information disclosed herein for the first time and ligated at restriction sites common to the clones to assemble a full-length clone encoding the VEGF. Alternatively, genomic libraries will provide the desired DNA. Once this DNA has been identified and isolated from the library, it is ligated into a replicable vector for further cloning or for expression.

In one example of a recombinant expression system, a polypeptide of the invention, e.g., a VEGF-encoding gene, etc., is expressed in a cell system by transformation with an expression vector comprising DNA encoding, e.g., the VEGF. In certain embodiments of the invention, it is preferable to transform host cells capable of accomplishing such processing so as to obtain the polypeptide in the culture medium or periplasm of the host cell, i.e., obtain a secreted molecule.

In some aspects of the invention, the Flt-1 agonist comprises a growth factor that selectively binds to and activates Flt-1. Several naturally occurring VEGF homologues that specifically bind to Flt-1 but not KDR have been identified, including without limiting to, placental growth factor (PIGF) and VEGF-B. PIGF has an amino acid sequence that shares 53% identity with the platelet-derived growth factor-like domain of VEGF. Park et al. J. Biol. Chem. 269:25646-54 (1994); Maglione et al. Oncogene 8:925-31 (1993). As with VEGF, different species of PIGF arise from alternative splicing of mRNA, and the protein exists in dimeric form. See, e.g., Park et al., supra. Both PIGF-1 and PIGF-2 bind to Flt-1 with high affinity, but neither is able to interact with KDR. See, e.g., Park et al., supra.

VEGF-B is produced as two isoforms (167 and 185 residues) that also appear to specifically bind Flt-1. Pepper et al. Proc. Natl. Acad. Sci. USA 95:11709-11714 (1998). Similar to the long forms of VEGF, VEGF-B is expressed as a membrane-bound protein that can be released in a soluble form after addition of heparin. VEGF-B and VEGF are also able to form heterodimers, when coexpressed. Olofsson et al. Proc. Natl. Acad. Sci. USA 93:2576-2581 (1996).

Compounds useful in the invention include small oraganic molecules that exert their modulating functions at the intracellular tyrosine kinase domain of the RTKs. In certain preferred embodiments, small molecule agonists are employed to stimulate tyrosine phosphorylation, thereby activating the corresponding signaling pathway.

Compounds useful in the invention include agonist antibodies. Antibodies of the present invention are typically specific against a receptor (such as Flt-1). In certain embodiments of the invention, antibodies of the invention include anti-Flt-1 antibodies. In one embodiment of the invention, the anti-Flt-1 antibody selectively binds to and modulate Flt-1, without affecting the KDR function. In one embodiment of the invention, the anti-Flt1 antibody is an agonist antibody.

### Uses

The invention provides methods for treatment of kidney disease, e.g., by promoting mesangial cell survival by administering an effective amount of VEGFR agonists. The survival promoting effects of the invention can be assessed either in vitro or in vivo, using methods known in the art and those described herein. For example, induction of collagen synthesis can be assessed (see, e.g., Amemiya, T., et al. Vascular endothelial growth factor activates MAP kinase and enhances collagen synthesis in human mesangial cells. Kidney Int 56:2055-2063 (1999)) and nitric oxide production can be monitored (see, e.g., Trachtman, H., et al. Effect of vascular endothelial growth factor on nitric oxide production by cultured rat mesangial cells. Biochem Biophys Res Commun 245:443-446 (1998)). Cell proliferation is assessed during culture using methods known in the art, including but not limited to, measuring the rate of DNA synthesis, trypan blue dye exclusion/hemacytometer counting, or flow cytometry. See also, e.g., Onozaki, A., et al. Rapid change of glucose concentration promotes mesangial cell proliferation via VEGF: inhibitory effects of thiazolidinedione. Biochem Biophys Res Commun 317:24-29 (2004).

In one aspect, the invention provides methods of using VEGFR agonists to upregulate or downregulate gene expression of factors that are important in regulating kidney activities, e.g., Table 2. Methods and techniques for detecting levels of mRNA expression or protein expression in target cells/tissues are known to those skilled in the art. For example, gene expression level can be detected by known nucleic acid hybridization assays, using probes capable of hybridizing to polynucleotides, under conditions suitable for the hybridization and subsequent detection and measurement. Methods useful for detecting gene expression include but not limited to southern hybridization (Southern J. Mol. Biol. 98:503-517 (1975)), northern hybridization (see, e.g., Freeman et al. Proc. Natl. Acad. Sci. USA 80:4094-4098 (1983)), restriction endonuclease mapping (Sambrook et al. (2001) Molecular Cloning, A Laboratory Manual, 3rd Ed. Cold Spring Harbor Laboratory Press, New York), RNase protection assays (Current Protocols in Molecular Biology, John Wiley and Sons, New York, 1997), DNA sequence analysis, and polymerase chain reaction amplification (PCR; U.S. Pat. Nos. 4,683,202, 4,683,195, and 4,889,818; Gyllenstein et al. Proc Natl. Acad. Sci. USA 85:7652-7657 (1988); Ochman et al. Genetics 120:621-623 (1988); and, Loh et al. Science 243:217-220 (1989) followed by Southern hybridization with probes specific for the gene, in various cell types. Other methods of amplification commonly known in the art can be employed. The stringency of the hybridization conditions for northern or Southern blot analysis can be manipulated to ensure detection of nucleic acids with the desired degree of relatedness to the specific probes used. The expression of gene in a cell or tissue sample can also be detected and quantified using in situ hybridization techniques according to, for example, Current Protocols in Molecular Biology, John Wiley and Sons, New York, 1997.

Protein levels can be detected by immunoassays using antibodies specific to protein. Various immunoassays known in the art can be used, including but not limited to competitive and non-competitive assay systems using techniques such as radioimmunoassay, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels), western blot analysis, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

### Antibodies

Antibodies of the invention include anti-VEGFR antibodies or antigen-binding fragments of VEGFR, or other antibodies described herein. Exemplary antibodies include, e.g., polyclonal, monoclonal, humanized, fragment, multispecific, heteroconjugated, multivalent, effector function, etc., antibodies. In certain embodiments of the invention, the antibody is an agonist antibody.

### Polyclonal Antibodies

The antibodies of the invention can comprise polyclonal antibodies. Methods of preparing polyclonal antibodies are known to the skilled artisan. For example, polyclonal antibodies against VEGFR are raised in animals by one or multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against VEGFR, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Typically, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that typically contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Typical myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against VEGFR. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In another embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Humanized and Human Antibodies

Antibodies of the invention can comprise humanized antibodies or human antibodies. A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a typical method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Human antibodies can also be produced using various techniques known in the art, including phage display libraries (Hoogenboom and Winter, J. Mol. Biol., 227:381 (1991); Marks et al., J. Mol. Biol., 222:581 (1991)). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K S, and Chiswell, D J., Cur Opin in Struct Biol 3:564-571 (1993). Several sources of V-gene segments can be used for phage display. For example, Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated, e.g., by essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. The techniques of Cole et al. and Boerner et al. are also available for the preparation of human monoclonal antibodies (Cole et al., Monoclonal Antibodies and Cancer Therapy, Alan R. Liss, p. 77 (1985) and Boerer et al., J. Immunol., 147(1):86-95 (1991)). Human antibodies may also be generated by in vitro activated B cells (see U.S. Pat. Nos. 5,567,610 and 5,229,275).

### Antibody Fragments

Antibody fragments are also included in the invention. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. SFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

### Multispecific Antibodies (e.g., Bispecific)

Antibodies of the invention also include, e.g., multispecific antibodies, which have binding specificities for at least two different antigens. While such molecules normally will only bind two antigens (i.e. bispecific antibodies, BsAbs), antibodies with additional specificities such as trispecific antibodies are encompassed by this expression when used herein. Examples of BsAbs include those with one arm directed against a cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; BsAbs with one arm which binds specifically to an antigen on a cell and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA); BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor:CD3 complex/anti-influenza, anti-FcγR/anti-HFV; BsAbs for tumor detection in vitro or in vivo such as anti-CEA/anti-EOTUBE, anti-CEA/anti-DPTA, anti-p185HER²/anti-hapten; BsAbs as vaccine adjuvants; and BsAbs as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase. Examples of trispecific antibodies include anti-CD3/anti-CD4/anti-CD37, anti-CD3/anti-CD5/anti-CD37 and anti-CD3/anti-CD8/anti-CD37. In one embodiment of the invention, a bispecific antibody is an anti-Flt1 agonist/anti-Integrin α-8. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the VEGF receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

### Heteroconjugate Antibodies

Bispecific antibodies include cross-linked or “heteroconjugate” antibodies, which are antibodies of the invention. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### Multivalent Antibodies

Antibodies of the invention include a multivalent antibody. A multivalent antibody may be internalized (and/or catabolized) faster than a bivalent antibody by a cell expressing an antigen to which the antibodies bind. The antibodies of the invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### Effector Function Engineering

It may be desirable to modify the antibody of the invention with respect to effector function, so as to enhance the effectiveness of the antibody in treating disease, for example. For example, a cysteine residue(s) may be introduced in the Fc region, thereby allowing interchain disulfide bond formation in this region. The homodimeric antibody thus generated may have improved internalization capability. See Caron et al., J. Exp Med. 176:1191-1195 (1992) and Shopes, B. J. Immunol. 148:2918-2922 (1992). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

### Liposomes and Nanoparticles

Polypeptides of the invention can be formulated in liposomes. For example, VEGFR modulators of the invention may also be formulated as immunoliposomes. Liposomes containing the polypeptide are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. Generally, the formulation and use of liposomes is known to those of skill in the art.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A polypeptide of the invention can be conjugated to the liposomes as described in Martin et al. J. Biol. Chem. 257: 286-288 (1982) (e.g., Fab′ fragments of an antibody) via a disulfide interchange reaction. Nanoparticles or nanocapsules can also be used to entrap the polypeptides of the invention. In one embodiment, a biodegradable polyalky-cyanoacrylate nanoparticles can be used with the polypeptides of the invention.

### Other Uses

The anti-VEGFR antibodies have various utilities. For example, anti-VEGFR antibodies may be used in diagnostic assays for VEGFR or fragments of VEGFR, e.g., detecting its expression in specific cells, tissues, or serum, for disease detection, e.g., of the disorders described herein, etc. In one embodiment, VEGFR antibodies are used for selecting the patient population for treatment with the methods provided herein. Various diagnostic assay techniques known in the art may be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. And Cytochem., 30:407 (1982).

Anti-VEGFR antibodies also are useful for the affinity purification of VEGFR from recombinant cell culture or natural sources. In this process, the antibodies against VEGFR are immobilized on a suitable support, such a Sephadex resin or filter paper, using methods well known in the art. The immobilized antibody then is contacted with a sample containing the VEGFR to be purified, and thereafter the support is washed with a suitable solvent that will remove substantially all the material in the sample except the VEGFR, which is bound to the immobilized antibody. Finally, the support is washed with another suitable solvent that will release the VEGFR from the antibody.

### Vectors, Host Cells and Recombinant Methods

The polypeptides of the invention can be produced recombinantly, using techniques and materials readily obtainable.

For recombinant production of a polypeptide of the invention, e.g., a polypeptide VEGFR modulating agent, the nucleic acid encoding it is isolated and inserted into a replicable vector for further cloning (amplification of the DNA) or for expression. DNA encoding the polypeptide of the invention is readily isolated and sequenced using conventional procedures. For example, a DNA encoding a monoclonal antibody is isolated and sequenced, e.g., by using oligonucleotide probes that are capable of binding specifically to genes encoding the heavy and light chains of the antibody. Many vectors are available. The vector components generally include, but are not limited to, one or more of the following: a signal sequence, an origin of replication, one or more marker genes, an enhancer element, a promoter, and a transcription termination sequence.

### Signal Sequence Component

Polypeptides of the invention may be produced recombinantly not only directly, but also as a fusion polypeptide with a heterologous polypeptide, which is typically a signal sequence or other polypeptide having a specific cleavage site at the N-terminus of the mature protein or polypeptide. The heterologous signal sequence selected typically is one that is recognized and processed (i.e., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the polypeptide of the invention.

### Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

### Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, typically primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding a polypeptide of the invention, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid Yrp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

### Promotor Component

Expression and cloning vectors usually contain a promoter that is recognized by the host organism and is operably linked to a nucleic acid encoding a polypeptide of the invention. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the polypeptide of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldyhyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldyhyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of polypeptides of the invention from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and typically Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the rous sarcoma virus long terminal repeat can be used as the promoter.

### Enhancer Element Component

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is typically located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide of the invention. One useful transcription termination component is the bovine growth hormone polyadenylation region. See WO94/11026 and the expression vector disclosed therein.

### Selection and Transformation of Host Cells

Suitable host cells for cloning or expressing DNA encoding the polypeptides of the invention in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Typically, the E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide of the invention-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptides of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for polypeptide of the invention production and cultured in conventional nutrient media modified as appropriate for inducing promoters, selecting transformants, or amplifying the genes encoding the desired sequences.

### Culturing the Host Cells

The host cells used to produce polypeptides of the invention may be cultured in a variety of media. Commercially available media such as Ham's F10 (Sigma), Minimal Essential Medium ((MEM), (Sigma), RPMI-1640 (Sigma), Dulbecco's Modified Eagle's Medium ((DMEM), Sigma), normal growth media for kidney cells, etc. are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polypeptide Purification

When using recombinant techniques, a polypeptide of the invention, e.g., a polypeptide VEGFR modulating agent, can be produced intracellularly, in the periplasmic space, or directly secreted into the medium. Polypeptides of the invention may be recovered from culture medium or from host cell lysates. If membrane-bound, it can be released from the membrane using a suitable detergent solution (e.g. Triton-X 100) or by enzymatic cleavage. Cells employed in expression of a polypeptide of the invention can be disrupted by various physical or chemical means, such as freeze-thaw cycling, sonication, mechanical disruption, or cell lysing agents.

It may be desired to purify a polypeptide of the invention from recombinant cell proteins or polypeptides. The following procedures are exemplary of suitable purification procedures: by fractionation on an ion-exchange column; ethanol precipitation; reverse phase HPLC; chromatography on silica, chromatography on beparin SEPHAROSE™ chromatography on an anion or cation exchange resin (such as a polyaspartic acid column, DEAE, etc.); chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides of the invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide of the invention produced.

For example, an antibody composition prepared from the cells can be purified using, for example, hydroxylapatite chromatography, gel electrophoresis, dialysis, and affinity chromatography, with affinity chromatography being the typical purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification, e.g., those indicated above, are also available depending on the antibody to be recovered. See also, Carter et al., Bio/Technology 10: 163-167 (1992) which describes a procedure for isolating antibodies which are secreted to the periplasmic space of E. coli.

### Covalent Modifications to Polypeptides of the Invention

Covalent modifications of a polypeptide of the invention, e.g., a polypeptide VEGFR modulating agent, etc.), are included within the scope of this invention. They may be made by chemical synthesis or by enzymatic or chemical cleavage of the polypeptide, if applicable. Other types of covalent modifications of the polypeptide are introduced into the molecule by reacting targeted amino acid residues of the polypeptide with an organic derivatizing agent that is capable of reacting with selected side chains or the N- or C-terminal residues, or by incorporating a modified amino acid or unnatural amino acid into the growing polypeptide chain, e.g., Ellman et al. Meth. Enzym. 202:301-336 (1991); Noren et al. Science 244:182 (1989); and, & US Patent applications 20030108885 and 20030082575.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is typically performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125, or 131, to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to a polypeptide of the invention, e.g., a polypeptide VEGFR modulating agent, etc. These procedures are advantageous in that they do not require production of the polypeptide in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981).

Removal of any carbohydrate moieties present on a polypeptide of the invention may be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al. Arch. Biochem. Biophys. 259:52 (1987) and by Edge et al. Anal. Biochem., 118:131 (1981). Enzymatic cleavage of carbohydrate moieties, e.g., on polypeptides (e.g., antibodies), can be achieved by the use of a variety of endo- and exo-glycosidases as described by Thotakura et al. Meth. Enzymol. 138:350 (1987).

Another type of covalent modification of a polypeptide of the invention comprises linking the polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

### Pharmaceutical Compositions

For in vivo uses according to the methods of the invention, a therapeutic compound of the invention is administered to a subject using methods and techniques known in the art and suitable for the particular use. In a preferred embodiment, the compound is administered in the form of pharmaceutical compositions at a pharmaceutically acceptable dosage.

In one aspect, the invention contemplates the use of protein preparations of the therapeutic protein agent for the administration of a therapeutic protein agent (e.g., recombinant protein preparations). In one aspect, the invention contemplates the use of mammalian cell preparations for the administration of a therapeutic protein agent (e.g., a polypeptide VEGFR modulating agent, etc.). The mammalian cells used herein have been transfected with the heterologous gene encoding the protein, as described in detail above. In one embodiment, the host cells used for the administration are CHO cells.

Therapeutic formulations of molecules of the invention, (such as VEGFR modulating agent, e.g., VEGF, VEGFR variant, VEGF variant (e.g., Flt1-sel or KDR-sel), VEGFR antibody, VEGFR small molecule modulator, etc.), used in accordance with the invention are prepared for storage by mixing a molecule, e.g., a polypeptide or small molecule, having the desired degree of purity with optional pharmaceutically acceptable carriers, excipients or stabilizers (Remington's Pharmaceutical Sciences 20th edition, Osol, A. Ed. (2000)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 20th edition, Osol, A. Ed. (2000). See also Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

In certain embodiments, the formulations to be used for in vivo administration are sterile. This is readily accomplished by filtration through sterile filtration membranes.

Sustained-release preparations may be prepared. Suitable examples of sustained-release preparations include semipermeable matrices of solid hydrophobic polymers containing a polypeptide of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), poly-lactic-coglycolic acid (PLGA) polymer, and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. See also, e.g., U.S. Pat. No. 6,699,501, describing capsules with polyelectrolyte covering.

It is further contemplated that a therapeutic protein agent of the invention (e.g., a VEGFR modulator, e.g., VEGF, VEGFR variant, VEGF variant (e.g., Flt1-sel or KDR-sel), VEGFR antibody, etc.) can be introduced to a subject by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 (1986)). The oligonucleotides can be modified to enhance their uptake, e.g. by substituting their negatively charged phosphodiester groups by uncharged groups. For general reviews of the methods of gene therapy, see, for example, Goldspiel et al. Clinical Pharmacy 12:488-505 (1993); Wu and Wu Biotherapy 3:87-95 (1991); Tolstoshev Ann. Rev. Pharmacol. Toxicol. 32:573-596 (1993); Mulligan Science 260:926-932 (1993); Morgan and Anderson Ann. Rev. Biochem. 62:191-217 (1993); and May TIBTECH 11:155-215 (1993). Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. For example, in vivo gene transfer techniques include but are not limited to, e.g., transfection with viral (typically retroviral) vectors and viral coat protein-liposome mediated transfection (Dzau et al., Trends in Biotechnology 11, 205-210 (1993)). For example, in vivo nucleic acid transfer techniques include transfection with viral vectors (such as adenovirus, Herpes simplex I virus, lentivirus, retrovirus, or adeno-associated virus) and lipid-based systems (useful lipids for lipid-mediated transfer of the gene are DOTMA, DOPE and DC-Chol, for example). Examples of using viral vectors in gene therapy can be found in Clowes et al. J. Clin. Invest. 93:644-651 (1994); Kiem et al. Blood 83:1467-1473 (1994); Salmons and Gunzberg Human Gene Therapy 4:129-141 (1993); Grossman and Wilson Curr. Opin. in Genetics and Devel. 3:110-114 (1993); Bout et al. Human Gene Therapy 5:3-10 (1994); Rosenfeld et al. Science 252:431-434 (1991); Rosenfeld et al. Cell 68:143-155 (1992); Mastrangeli et al. J. Clin. Invest. 91:225-234 (1993); and Walsh et al. Proc. Soc. Exp. Biol. Med. 204:289-300 (1993).

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

For example, viral or nonviral vectors for gene therapy as well as genetically modified renal cells have been used for the delivery of foreign genes in the kidney. Various vectors were injected into renal cells through different routes, via intraarterial, intraureteral or intraparenchymal injections (Bosch R J et al., (1993) Exp Nephrol 1: 49-54; and, Ye X et al., (2001) Hum Gene Ther 12: 141-148). The major limitation of intraparenchymal injection was that it caused some renal injury. The delivery of a transgene to the kidney ex vivo prior to transplantation into a recipient could also be used in some cases.

### Dosage and Administration

Dosages and desired drug concentrations of pharmaceutical compositions of the invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

Depending on the type and severity of the disease, about 1 μg/kg to 50 mg/kg (e.g. 0.1-20 mg/kg) of VEGFR modulator is an initial candidate dosage for administration to the patient, whether, for example, by one or more separate administrations, or by continuous infusion. When in vivo administration of a VEGFR modulator is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue. For repeated administrations over several days or longer, depending on the condition, the treatment is sustained until a desired suppression of disease symptoms occurs. However, other dosage regimens may be useful. Typically, the clinician will administered a molecule(s) of the invention until a dosage(s) is reached that provides the required biological effect. The progress of the therapy of the invention is easily monitored by conventional techniques and assays.

The therapeutic composition of the invention can be administered by any suitable means, including but not limited to, parenteral, subcutaneous, intraperitoneal, intrapulmonary, intracerobrospinal, subcutaneous, intra-articular, intrasynovial, intrathecal, oral, topical, and intranasal administration. Parenteral infusions include intramuscular, intravenous, intraarterial, intraperitoneal, or subcutaneous administration. In addition, the therapeutic composition is suitably administered by pulse infusion, particularly with declining doses of the modulator. In certain embodiments, the therapeutic composition is given by injections, e.g., intravenous or subcutaneous injections, depending in part on whether the administration is brief or chronic.

Use of multiple agents is also included in the invention. As described herein, VEGFR modulator can be combined with one or more therapeutic agents. The combined administration includes coadministration, using separate formulations or a single pharmaceutical formulation, and consecutive administration in either order. For example, an VEGFR agonist may precede, follow, alternate with administration of the additional therapeutic agent (e.g., an angiogenic agent), or may be given simultaneously therewith. In one embodiment, there is a time period while both (or all) active agents simultaneously exert their biological activities.

For the prevention or treatment of disease, the appropriate dosage of VEGFR modulator, will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments. In a combination therapy regimen, the compositions of the invention are administered in a therapeutically effective amount or a therapeutically synergistic amount. As used herein, a therapeutically effective amount is such that co-administration of VEGFR modulator, and one or more other therapeutic agents, or administration of a composition of the invention, results in reduction or inhibition of the targeting disease or condition. A therapeutically synergistic amount is that amount of VEGFR modulator, and one or more other therapeutic agents, e.g., described herein, necessary to synergistically or significantly reduce or eliminate conditions or symptoms associated with a particular disease.

### Articles of Manufacture

In another embodiment of the invention, an article of manufacture containing materials useful for the methods and treatment of the disorders described above is provided. The article of manufacture comprises a container, a label and a package insert. Suitable containers include, for example, bottles, vials, syringes, etc. The containers may be formed from a variety of materials such as glass or plastic. The container holds a composition which is effective for treating the kidney disease and may have a sterile access port (for example the container may be an intravenous solution bag or a vial having a stopper pierceable by a hypodermic injection needle). At least one active agent in the composition is a VEGFR modulator. The label on, or associated with, the container indicates that the composition is used for treating kidney disease. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, and syringes. Optionally, a set of instructions, generally written instructions, is included, which relates to the use and dosage of VEGFR modulator for a disorder described herein. The instructions included with the kit generally include information as to dosage, dosing schedule, and route of administration for the treatment the disorder. The containers of VEGFR modulator may be unit doses, bulk packages (e.g., multi-dose packages), or sub-unit doses.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 1

### Identification of a Novel Autocrine Regulatory Loop by VEGF-A in Kidney Mesangial Cells Mediated by Flt1/VEGFR-1

We generated transgenic mice whereby the VEGF gene was ablated in cells expressing the VEGF receptor-1 (Flt1/VEGFR-1). We found that VEGF-A gene ablation in kidney mesangial cells resulted in progressive renal failure characterized by proteinuria, glomerular sclerosis, hypertension and death in mice aged 1-3 months. Affected glomeruli displayed reduced VEGF-A expression in podocytes and increased numbers of inflammatory cells, immune complex depositions and complement activation. Interference with the autocrine loop in mesangial cells induces distinct renal changes reminiscent of a subset of human kidney pathologies associated with reduced renal VEGF levels. In vitro, VEGF-A- and Flt-1-deficient mesangial cells displayed decreased cell survival and a shift in gene expression towards ECM synthesis and reduced matrix degradation. These findings identify a novel autocrine signaling loop between VEGF-A and VEGFR-1 regulating ECM production and VEGF expression in podocytes. Stimulation of VEGFR1 in kidney cells, e.g., mesangial cells, can be a therapeutic strategy for the treatment of progressive glomerulosclerosis associated with decreased VEGF-A levels.

Flt-1: fins-like tyrosine kinase; GBM: glomerular basement membrane; VEGF: vascular endothelial growth factor; VEGFR: VEGF receptor; VEGFR-1: VEGF receptor; Flt-1: fins-like tyrosine kinase; VEGFR-2: VEGF receptor, KDR (or Flk1); WT: wild-type; WT-1: Wilm's Tumor nuclear protein-1; ECM: extracellular matrix

### Materials and Methods

Generation of VEGF-loxP and Flt1-Cre mice and breeding to the ROSA26 reporter strain: VEGF-loxP mice were generated as previously described (see, e.g., Gerber, H. P., et al. VEGF is required for growth and survival in neonatal mice. Development 126:1149-1159(1999a)). Briefly, in VEGF-loxP mice, exon 3 of VEGF is flanked by loxP sites, resulting in a null VEGF allele in cells that undergo loxP recombination. VEGF-loxP mice were bred with Flt1-Cre mice in which a 3.1 kb fragment of the Flt1 promotor (see, e.g., Gerber, H. P., et al. Differential transcriptional regulation of the two vascular endothelial growth factor receptor genes. Flt-1 but not Flk-1/KDR, is up-regulated by hypoxia. J Biol Chem 272:23659-23667 (1997)) drives expression of Cre-recombinase. Flt1-Cre mice were generated by microinjecting a construct containing a 3.1 kb fragment of the Flt1 promotor, driving expression of Cre-recombinase, into mouse egg pronuclei as described previously. See, Hogan, B., et al. (eds.). Manipulating the mouse embryo, (Cold Spring Harbor Laborator Press, 1994). To monitor expression of Cre-recombinase, Flt-CRE⁺; VEGF-^{loxP/loxP)} or Flt-Cre+ mice were crossed to the ROSA26 reporter strain (see, e.g., Mao, X., et al. Improved reporter strain for monitoring Cre recombinase-mediated DNA excisions in mice. Proc Natl Acad Sci USA 96, 5037-5042 (1999)), whereby the ubiquitous expression of β-galactosidase is inhibited by transcriptional/translational terminating signals. This ‘stopper-fragment’ is flanked by loxP sites and undergoes Cre-mediated excision resulting in expression of the β-galactosidase gene in cells that express Cre-recombinase.

Generation of Flt1-loxP mice: A 16-kb genomic Flt-1 DNA clone encompassing exon 1 of the murine Flt1 gene locus was isolated following screening of a bacterial artificial chromosome library using the following primers: A 1.4 kb HindIII genomic DNA fragment spanning 3.0 to 1.6 kb upstream of the Flt1 translation initiation codon was excised and blunt-end cloned into the NotI site of TNLOX1-3 targeting vector. Subsequently, a 2.0 kb HindIII/BstXI genomic DNA fragment was cloned by blunt-end ligation into the unique AscI site of TNLOX1-3, downstream of the PGK-neo^{R} cassette and immediately 5′ of LoxP3. This 2.0 kb fragment included a region of the Flt1 gene promotor, transcription start site and exon 1 of the Flt1 gene. Finally, a 2.0 kb BstXI/BsmI genomic DNA fragment was blunt-ended and cloned into the PmeI site immediately 3′ of the third loxP site, to generate the targeting vector denoted TKNeoFlt1-1. TKNeoFlt1-1 was sequenced and subjected to restriction endonuclease digestion to verify the sequence and orientation of the loxP sites and genomic DNA inserts.

The targeting vector was linearised by SalI digestion and 20 μg electroporated into TCL1 and R1 ES cells that are derived from the 129Sv strain. ES cells and mouse embryonic fibroblasts were maintained in culture in the presence of murine leukemia inhibitory factor (LIF) as previously described (see, e.g., Gerber, H. P., et al. VEGF is required for growth and survival in neonatal mice. Development 126:1149-1159 (1999a)). ES cells were subjected to positive selection with G418 (400 μg/ml) 24 hours after electroporation and after nine days of this selection, individual colonies were picked, grown and screened for positive recombination events by Southern blot analysis. Genomic DNA from resistant clones was digested with either EcoRI (for analysis of the 5′ end of the targeting event) or with both HindIII and KpnI (for analysis at the 3′ end of the targeted genomic region). The probes used to screen the 5′ and 3′ ends of the targeted region were generated by PCR using the following primer pairs: 5′ Probe (639 nts): Flt-LOX.1123F (GAT GGC CTT GAG TAT ATC CTG (SEQ ID NO:1)) and Flt-LOX.1762R (CAG CTC TGG ACT CCA GCT TGC (SEQ ID NO:2)); 3′ Probe (834 nts): Flt-LOX.9733F (GGA AAC TAT GTG GCT GAT CTC (SEQ ID NO:3)) and Flt-LOX.10567R (GTG AGA GCC AAG ATC GAG GAG (SEQ ID NO:4)). Two independent ES cell clones, designated #15 and #F7 were identified as homologous recombinants and transiently transfected with an expression vector encoding Cre-recombinase (pMC-Cre) as described previously (see, e.g., Gerber, H. P., et al. VEGF is required for growth and survival in neonatal mice. Development 126:1149-1159 (1999a)). The transfected ES clones were picked to obtain individual colonies and screened by Southern blot and PCR for deletion of the PGK-neo^{R} cassette and recombination between LoxP1 and LoxP2. In addition to Southern blot analysis, the selected colonies were also analysed by PCR using the primers Flt-LOX.236F (TAG ACT CTG CGC GCC ATA ACT (SEQ ID NO:5)) and Flt-LOX.2629R (CAC TAA GAA GGC AGA GGC CAA (SEQ ID NO:6)). Flt-LOX.236F anneals to DNA immediately 5′ and overlapping with the first 6 nucleotides of LoxP3, and used in combination with Flt-LOX.2629R (that is homologous to DNA downstream of the 3′ arm of homology). will only generate a PCR product from DNA containing LoxP3. These primers were used to further confirm that the third loxP site had not undergone recombination.

One ES cell clone derived from #15 and #F7, in which the PGK-neo^{R} cassette was removed (denoted #15.C1.H1 and #F7.A.E11 respectively), was injected into the blastocoele cavity of 3.5 day C57B1/6J blastocysts (see, e.g., Hogan et al., et al. (eds.). Manipulating the mouse embryo, (Cold Spring Harbor Laborator Press) (1994)). Chimeric males were mated with C57B1/6J female mice and the offspring screened for germline transmission by PCR analysis to detect LoxP1/2 and LoxP3. The PCR primers used to screen for the presence of LoxP1/2 were Flt-LOX.1335F (CCT GCA TGA TTC CTG ATT GGA (SEQ ID NO:7)) and Flt-LOX.3207R (GCC TAA GCT CAC CTG CGG (SEQ ID NO: 8)). The PCR primers used to screen for the presence of LoxP3 were Flt-LOX.236F and Flt-LOX.2629R. Flt1-LoxP(+/−) mice were then crossed to generate Flt1-LoxP(−/−) that do not carry a floxed Flt1 allele, Flt1-LoxP(+/−) which carry a single Flt1 allele that is floxed, and Flt1-LoxP(+/+) mice in which both alleles of Flt1 contain loxP sites. Flt1-loxP mice were typically genotyped by PCR using the Flt-LOX.1335F and Flt-LOX.3207R oligonucleotides.

In situ hybridization: In situ hybridization for VEGF and TGF-β was carried using antisense and sense probes generated by PCR amplification using primers specific for murine TGF-β (Forward: 5′-CACCGCGACTCCTGCTGCTTT (SEQ ID NO: 9); Reverse:5′-GGGGGTTCGGGCACTGCTT (SEQ ID NO: 10); probe size: 609 nt) and rat VEGF (Forward: 5′-CAACGTCACTATGCAGATCATGCG (SEQ ID NO: 11); Reverse: 5′-TCACCGCCTTGGCTTGTCA (SEQ ID NO:12); probe size: 348 nt). Kidney tissue was excised from 7.5 week old mice, fixed in 4% formalin and paraffin-embedded. Sections 5 μm thick were deparaffinized, deproteinated in 4 μg/ml of proteinase K for 30 min at 37° C. and further processed for in situ hybridization as previously described (see, e.g., Gerber, H. P., et al. VEGF couples hypertrophic cartilage remodeling, ossification and angiogenesis during endochondral bone formation. Nat Med 5:623-628(1999b)). ³³P-UTP labeled sense and antisense probes were hybridized to the sections at 55° C. overnight. Unhybridized probe was removed by incubation in 20 μg/ml RNase A for 30 min at 37° C., followed by a high stringency wash at 55° C. in 0.1× standard saline citrate (SSC) for 2 hours and dehydration through graded ethanols. The slides were dipped in NBT2 nuclear track emulsion (Eastman Kodak), exposed in sealed plastic slide boxes containing dessicant for 4-6 weeks at 4° C., developed and counterstained with hematoxylin and eosin (H & E).

Electron microscopy: Pieces of cortical kidney tissue from 4 to 5 week old VEGF-loxP, Flt1-Cre mice were fixed overnight at 4° C. in 2% formaldehyde, 2.5% glutaraldehyde in 0.1M cacodylate buffer. After washing, the samples were postfixed in aqueous 1% osmium for 2 hours, washed in water, dehydrated through graded ethanols and propylene oxide, and embedded in EPONATE 12 (Ted Pella, Inc. Redding, Ca). Ultra-thin sections were cut on a Reichert Ultracut UCT microtome, counterstained with uranyl acetate and lead citrate and examined in a Philips CM12 transmission electron microscope at 80 kV. Images were captured with a GATAN Retractable Multiscan digital camera.

LacZ staining; To LacZ stain whole embryonic day 9.5 embryos or tissue from 1 week old mice, the tissues were dissected in phosphate buffered saline (PBS) and fixed with 4% paraformaldehyde (PFA) in PBS for 1 hour at 4° C. After three thirty minute washes in rinse buffer (5 mM ethylene glycol-bis(aminoethylether)-tetraacetic acid (EGTA), 0.01% deoxycholate, 0.02% NP-40, 2 mM MgCl₂ in PBS), embryos were incubated overnight at 37° C. in staining solution (rinse buffer containing 5 mM K₃Fe(CN)₆, 5 mM K₄Fe(CN)₆, 1 mg/ml 5-bromo-4-chloro-3-indolyl-β-D-galactoside (X-gal)). Tissues were then post-fixed in 4% PFA in PBS for 30 mins at 4° C., transferred into 70% ethanol and photographed using a Leica MZFLIII dissecting microscope, SPOT digital camera and SPOT Advanced photographic software or processed for paraffin embedding and sectioning.

Histological analysis and immunocytochemistry: For histological analysis, tissues were fixed in 10% neutral buffered formalin for 12 to 16 hours, transferred to 70% ethanol and paraffin embedded. 5 μm sections were cut using a microtome (Leica Microsystems, Wetzlar, Germany) and stained with hematoxylin and eosin (H&E). Paraffin embedded sections were analyzed by immunohistochemistry, using antibodies raised to Cre-recombinase (EMD Biosciences.Novagen, San Diego, Calif.), CD31 (MEC 13.3, BD Biosciences Pharmingen, San Diego, Calif.) that detects all endothelial cells, VEGFR-2/Flk-1 (MALK-1, Genentech, Inc. South San Francisco, Calif.) that primarily labels non-arterial vascular endothelium, and alpha smooth muscle actin (DakoCytomation California, Inc., Carpinteria, Calif.) that detects developing and activated mesangial cells and smooth muscle cells. To detect extracellular matrix components, antibodies raised against collagen IV (Chemicon Internation, Temecula, Calif.) and laminin (Chemicon) were used. Staining was performed essentially as described previously (see, e.g., Gerber, H. P., et al. VEGF is required for growth and survival in neonatal mice. Development 126:1149-1159 (1999a)).

For immunofluorescence studies, excised kidneys were dissected longitudinally, embedded in O.C.T. compound (Tissue Tek, Sakura Finetek U.S.A., Inc., Torrance, Calif.) and sectioned at 5 to 10 μm. To identify glomerular cell types that express mouse VEGF, frozen sections were incubated with a humanized monoclonal antibody recognizing mouse VEGF (α-VEGF, Genentech Inc.) at a concentration of 20 μg/ml, in combination with either integrin α8 affinity-purified rabbit antiserum (at 1/200 to detect mesangial cells, a kind gift from Ulrich Muller, The Scripps Research Institute, La Jolla, Calif.), rat α-mouse CD31 (BD Pharmingen) (to detect endothelial cells) or rabbit α-mouse Wilm's Tumor nuclear protein (WT-1; Santa Cruz Biotechnology Inc., 2 μg/ml) (to detect podocytes). Sections were subsequently washed and incubated with AlexaFluor-594 conjugated goat anti-human IgG and either AlexaFluor-488 conjugated goat anti-rat or goat anti-rabbit IgG secondary antibodies at 4 μg/ml (Invitrogen). To identify glomerular cell types expressing the Flt1-Cre transgene, frozen sections were incubated with anti-beta-galactosidase (Rockland Immunochemicals Inc.; 200 μg/ml), followed by AlexaFluor-594 conjugated goat anti-rabbit IgG. WT-1 antibody and anti-Integrin α8 were labeled with AlexaFluor-488 using the Zenon labeling technique for rabbit IgG (Invitrogen) according to the manufacturer. CD31 antibody, or the AlexaFluor-488-labelled α-WT-1 or anti-Integrin a8 were then applied to the washed sections. In this study, anti-beta-Gal staining reflects Flt1-Cre transgene expression during any or all stages of development as Cre-recombinase mediated excision irreversibly removes suppression of the otherwise ubiquitously expressed ROSA26 gene promoter that drives anti-beta-Gal expression in these mice (Mao et. al., Improved reporter strain for monitoring Cre recombinase-mediated DNA excisions in mice. Proc Natl Acad Sci USA 96:5037-42 (1999)).

To detect immunoglobulin deposits, a series of fluorescein isothiocyanate (FITC)-conjugated rat monoclonal antibodies specific for each class and isotype of mouse immunoglobulin (Ig) (BD Biosciences Pharmingen, San Jose, Calif.) were incubated on blocked frozen sections for 1.5 hours at a concentration of 4 μg/ml. Ig deposition was confirmed using affinity purified FITC-conjugated polyclonal antibodies raised to mouse Igs of specific class and isotype (Southern Biotechnology Associates, Birmingham, Ala.). C1q, C3 and C4 components of the mouse complement system were detected using rat monoclonal antibodies (HyCult Biotechnology b.v., Uden, The Netherlands) at 5 μg/ml and AlexaFluor-594-conjugated goat-anti-rat-IgG (Invitrogen) as the secondary reagent.

Cells of the T-cell and monocyte-macrophages lineage were identified using antibodies raised to CD4 (BD Biosciences, Pharmingen, San Jose, Calif.) and F4/80 (Serotec Inc., Raleigh, N.C.) respectively and standard staining protocols. For all immunocytochemical studies, negative controls were included by substituting primary antibodies with purified Igs, matched to the concentration, species and isotype of the omitted primary antibody.

To detect hypoxia in mouse kidneys, 4 week old Flt1-Cre− and Flt1-Cre+; VEGF(loxP/loxP) mice were injected intraperitoneally with pimonidazole hydrochloride (Hypoxyprobe™-1, Chemicon International, Temecula, Calif., 60 mg/kg). One hour following injection, mice were euthanised by cervical dislocation, kidneys excised and fixed overnight in 10% formalin, then dehydrated and embedded in paraffin. 5 μm paraffin embedded sections were then processed and stained with Hypoxyprobe™-1Mab1 as described by the Hypoxyprobe™-1 Kit manufacturer (Chemicon International), excluding the streptavidin peroxidase incubation that was substituted with streptavidin-AlexaFluor594 (Invitrogen) to allow for fluorescent detection. Mouse IgG 1 matched in concentration to that used for Hypoxyprobe™-Mab1 was used as an isotype control for non-specific staining of the primary antibody. As an additional negative control, kidney sections isolated from mice that were not injected with Hypoxyprobe™-1 were incubated with Hypoxyprobe™-Mab1.

Real-time quantitative RT-PCR analysis; RNA was isolated from tissues of 5 to 7.5 week old mice using the STAT 60 method (TEL-TEST “B”, Friendswood, Tex.) and purified on Rneasy Quick spin columns (Qiagen, Valencia, Calif.). Real-time quantitative RT-PCR analysis was performed as previously described (see, e.g., Gerber, H. P., et al. Complete inhibition of rhabdomyosarcoma xenograft growth and neovascularization requires blockade of both tumor and host vascular endothelial growth factor. Cancer Res 60:6253-6258 (2000)) using 100 ng of total RNA, Applied Biosystems RT-PCR reagents and a Model 7700 Sequence Detector in 96-well format (Applied Biosystems). RT-PCR conditions were 30 min at 48° C., 10 min at 95° C., and 40 cycles of 30 s at 95° C. and 90 s at 60° C. The results were analysed using Sequence Detection Software (Applied Biosystems) and statistical analysis by ANOVA was performed using StatView software (SAS Institute Inc., Cary, N.C.). Relative RNA equivalents for each sample were obtained by standardizing to glyceraldehydes-3-dehydrogenase (GAPDH) levels.

Serum chemistry and hematological parameters: 7.5 week old mice were euthanised by CO₂ inhalation and blood collected by cardiac puncture into ethylenediaminetetraacetic acid (EDTA)-coated tubes (Microtainer, Becton Dickinson and Company, Franklin Lakes, N.J.). Hematological cell counts were measured using a Cell Dyn 3700 (Abbott Laboratories, Abbott Park, Ill.). Serum was obtained by collection into serum-separator tubes (Microtainer, Becton Dickinson and Company, Franklin Lakes, N.J.) and serum parameters measured using a Roche Cobas Integra 400 instrument (Roche Diagnostics, Indianapolis, Ind.).

Urine Analysis: Urine was collected passively from mice aged 4 to 5 weeks. Representative urine samples from mice of each genotype were tested for the presence of protein, blood, glucose and ketones using urine test strips (Chemstrip 10 with SG, Roche Diagnostics Corp., Indianapolis, Ind., USA). Proteinuria was further confirmed by loading 1 ul of urine onto a 4-20% gradient tris/glycine gel (Invitrogen Corporation, Carlsbad, Calif.) and subjecting to SDS-PAGE, followed by silver staining or Western blotting for mouse albumin using affinity-purified goat anti-serum at 200 μg/ml (Bethyl Laboratories Inc., Montgomery, Tex.).

Measurement of mean arterial blood pressure: Mice were anesthetized with isoflurance inhalation to effect (Aerrane, Baxter Caribe Inc.). Through a ventral midline incision made in the neck, a catheter (polyethylene tubing, PE-10, Becton-Dickinson) was placed in the right common carotid artery and secured in place with silk suture. Blood pressure measurements were collected digitally for 15 minutes using AcqKnowledge hardware and software (Biopac Systems, Inc., Santa Barbara, Calif.).

Statistical Analyses: All statistical analyses, excluding analysis of complementary DNA microarray data (see separate section), were performed by analysis of variance (ANOVA) using StatView software (SAS Institute Inc., U.S.A.) unless otherwise stated.

Isolation of Glomeruli and Mesangial Cell Culture: Mouse glomeruli were isolated according to the method of (Takemoto et al., A new method for large scale isolation of kidney glomeruli from mice. Am J Pathol 161:799-805 (2002)) and plated onto dishes coated with 20 μg/mL fibronectin (Sigma Corp, St Louis, Mo.) in mesangial cell medium (Dulbecco's Modified Eagle Medium (DMEM), 20% fetal calf serum (FCS), 2 mM glutamine, 100 U/mL penicillin, 100 μg/mL streptomycin). Glomeruli were incubated in a humidified atmosphere of 5% CO₂, 95% air at 37° C. for 6-7 days during which time the glomeruli adhered to the plate and a confluent monolayer of cells covered the dish. Using this technique, homogeneous cultures of glomerular mesangial cells were obtained that could be passaged at least five times. The glomerular mesangial cell morphology appeared consistent with published reports of mesangial cells using light microscopy (Mene et al., Phospholipids in signal transduction of mesangial cells. Am J Physiol 256:F375-386 (1989)). The glomerular mesangial cells immunostained positive for α-vimentin (DakoCytomation) and anti-myosin (Zymed Laboratories, Inc, South San Francisco, Calif.), and were negative for CD31 (BD Biosciences Pharmingen, San Jose, Calif.) and acetylated low density lipoprotein uptake (Biomedical Technologies, Inc, Stoughton, Mass.). In addition, all mesangial cells were cultured in D-valine substituted minimum essential medium (MEM) for at least 3 days which blocks the growth of fibroblasts in vitro (Gilbert and Migeon, (1975). D-valine as a selective agent for normal human and rodent epithelial cells in culture. Cell 5:11-17).

Glomerular mesangial cell cultures were established from VEGF(loxP/loxP) and Flt1(loxP/loxP) mice and WT mice from the same colony that did not carry genomic loxP sites. For mesangial cell survival studies, mesangial cells were plated into six-well dishes at a density of 10⁵ cells/well, and incubated overnight in mesangial cell medium containing 20% FCS. The medium was then aspirated and replaced with serum-free medium containing adenovirus expressing either LacZ (Ad-LacZ) as a control, or Cre-recombinase (Ad-Cre) that induces recombination between loxP sites and results in VEGF or Flt1 gene ablation in VEGF(loxP/loxP) and Flt1(loxP/loxP) cells respectively. Adenovirus was used at a multiplicity of infection (MOI) of 1000 in the survival studies. A neutralizing anti-murine VEGF antibody (α-VEGF, G6-23-IgG) (Genentech, Inc. South San Francisco, Calif.) or a control isotype matched antibody (Control IgG) was added to the medium at 10 μg/mL and with Ad-LacZ at MOI of 1000. Four days following addition of adenovirus, remaining adherent cells were washed with PBS, trypsinized, and counted using a Z2 Coulter Particle Count and Size Analyzer (Beckman Coulter, Inc. Fullerton, Calif.). Mesangial cells were isolated from mice of each genotype (n=5-7 mice per genotype). Between two and five replicate wells were counted for each virus in each mouse from which cells were derived. The cell count ratio between Ad-LacZ and Ad-Cre treatments was calculated and normalized as a percentage of the value obtained for the control group.

Complementary DNA Microarrays: Mesangial cells were prepared from 4 WT and from 4 VEGF(loxP/loxP) mice. At passage two or three, the mesangial cells were cultured in serum-free medium containing Ad-LacZ or Ad-Cre at MOI of 100 for five days. RNA was isolated using the STAT60 method and Rneasy Quick Spin Columns as described in the ‘Real time quantitative RT-PCR’ section. The methods for preparation of complementary RNA (cRNA) and hybridization/scanning of the arrays were provided by Affymetrix (Affymetrix, Inc. Santa Clara, Calif.). Five μg total RNA was converted into double-stranded cDNA using a cDNA synthesis kit (SuperScript Choice, GIBCO/BRL, Grand Island, N.Y.) and a T7-(dT)₂₄ oligomer primer (Biosearch Technologies, Inc, Novato, Calif., Custom Synthesis). Double-stranded cDNA was purified on an affinity resin (Sample Cleanup Module Kit, Affymetrix, Inc. Santa Clara, Calif.) and by ethanol precipitation. After second-strand synthesis, labeled cRNA was generated from the cDNA sample by using a T7 RNA polymerase and biotin-labeled nucleotide in an in vitro transcription reaction (Enzo Biochem, Inc. Farmingdale, N.Y.). The labeled cRNA was purified on an affinity resin (sample cleanup module kit, Affymetrix). The amount of labeled cRNA was determined by measuring absorbance at 260 nm and using the convention that 1 OD at 260 nm corresponds to 40 μg/ml of RNA. Twenty μg of cRNA was fragmented by incubating at 94° C. for 30 minutes in 40 mM Tris-acetate (pH 8.1), 100 mM potassium acetate, and 30 mM magnesium acetate. Samples were then hybridized to Mouse Genome 430 2.0 arrays at 45° C. for 19 hours in a rotisserie oven set at 60 rpm. Arrays were washed, stained, and scanned in the Affymetrix Fluidics station and scanner. Data analysis was performed using the Affymetrix GeneChip Analysis software. Gene expression was summarized by Affymetrix MAS 5.0 signal values, which were analyzed on the logarithmic scale. An analysis of variance was applied by considering virus effects (Ad-LacZ or Ad-Cre), genotype effects (WT or VEGF(loxP/loxP)), and the effect of VEGF gene ablation for each probe set. The average fold change in gene expression from Ad-LacZ to Ad-Cre in the VEGF(loxP/loxP) cells versus the corresponding fold change in the WT cells, the strength of the evidence for gene expression difference (p value from t-test) and the minimum absolute signal of the gene expression were used as a combination of criteria to screen significantly affected probe sets by gene ablation effects. These criteria were set at a minimum fold change of 2-fold, a p value<0.05, and an absolute signal at >50. Changes in gene expression between WT and VEGF-deficient mesangial cells were compared.

To classify significantly dysregulated genes according to the gene ontology classifications provided by Affymetrix, we obtained the 9 Aug. 2004 version of the Gene Ontology (GO) hierarchies. The NetAffy database was used to associate each Affymetrix probe set with LocusLink gene identifications. The loc2go file was downloaded from NCBI LocusLink website (available on the internet at ftp://ftp.ncbi.nih.gov/refseq/LocusLink/), which was used to associate genes and GO concepts. The distribution of gene associations was computed on GO hierarchies by using the entire set of genes with GO annotations, and the set of genes affected by VEGF gene ablation. For each GO concept, a two-by-two contingency table was generated that represented presence or absence of the GO concept versus presence or absence of gene ablation effects. A chi-square analysis of association was performed to determine statistical significance. The odds ratio was computed by dividing the observed number of knockout-affecting genes for the GO concept by the expected number. A 95% confidence interval for the odds ratio was obtained from 1000 bootstrap samples.

Microarray data was also analyzed through the use of Ingenuity Pathways Analysis (IPA; Ingenuity® Systems, www.ingenuity.com). Identifiers for probes whose expression was significantly differentially regulated (p-value<4e-3) were loaded into the application where they were mapped to genes. The genes to which these probes mapped were used to generate molecular networks using information contained in the Ingenuity Pathways Knowledge Base (IPKB). For the functional analysis, these same genes were associated with biological functions and/or diseases using the IPKB. The Fischer exact test was used to calculate a p-value determining the probability that each biological function and/or disease assigned to that data set is due to chance alone.

### Results

### Flt1-Cre Transgene Expression During Development Recapitulates Endogenous Flt1 Expression

A 3.1 kb promoter fragment of the Flt1 gene was previously identified and characterized to be sufficient to mediate increased reporter gene expression in transiently transfected endothelial cells or Hep3 B cells exposed to hypoxic conditions (see, e.g., Gerber, H. P., et al. Differential transcriptional regulation of the two vascular endothelial growth factor receptor genes. Flt-1, but not Flk-1/KDR, is up-regulated by hypoxia. J Biol Chem 272:23659-23667 (1997)). A construct consisting of the same 3.1 kb Flt-1 promotor fragment was inserted upstream of the Cre− recombinase gene was used to generate transgenic mice. The strain with highest LacZ expression in adult kidneys was selected for detailed transgene expression analysis. Whole mount staining of transgenic embryos on day 9.5 day revealed a vascular expression pattern consistent with endogenous Flt1 gene expression. See also, e.g., Fong, G. H., et al. Regulation of flt-1 expression during mouse embryogenesis suggests a role in the establishment of vascular endothelium. Developmental Dynamics 207:1-10 (1996). In newborn mice, LacZ positive cells were present in a variety of tissues, including heart, spleen, lung, testis, and skin. Consistent with previous reports describing Flt-1 expression in the kidney endothelium and mesangial cells (see, e.g., Takahashi, T., et al. Protein tyrosine kinases expressed in glomeruli and cultured glomerular cells: Flt-1 and VEGF expression in renal mesangial cells. Biochem Biophys Res Commun 209:218-226 (1995)), LacZ positive cells were found in peritubular endothelial cells and cells within the central region of kidney glomerulus, where endothelial and mesangial cells are typically located in a Flt1-Cre⁺;ROSA⁺ mouse aged 5 days.

### Flt-CRE⁺; VEGF^{(loxP/loxP)} Mice Develop Glomerulonephritis and Succumb to End-Stage Renal Failure at Age 4-12 Weeks.

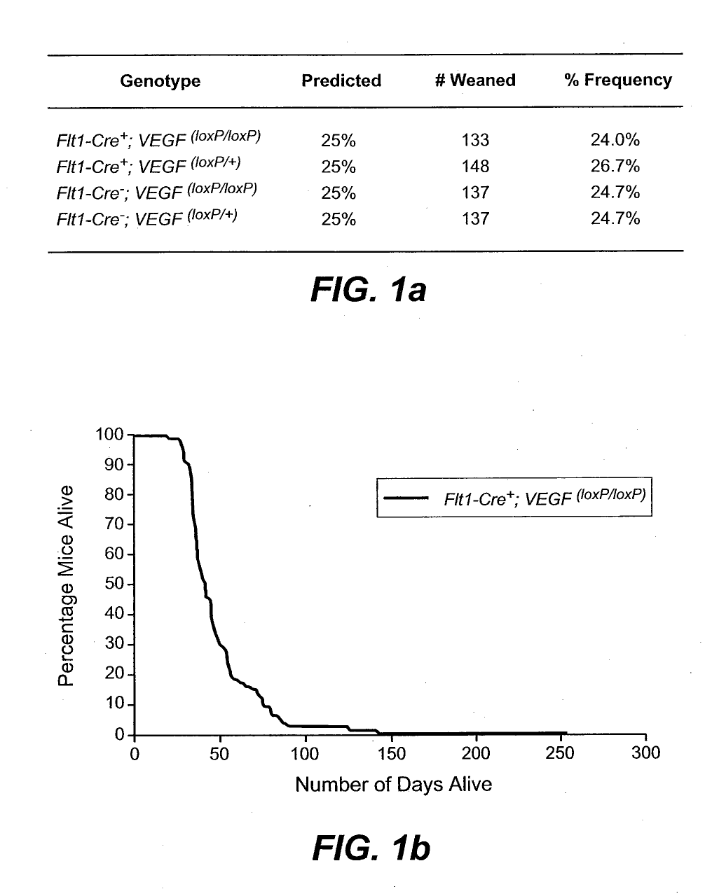

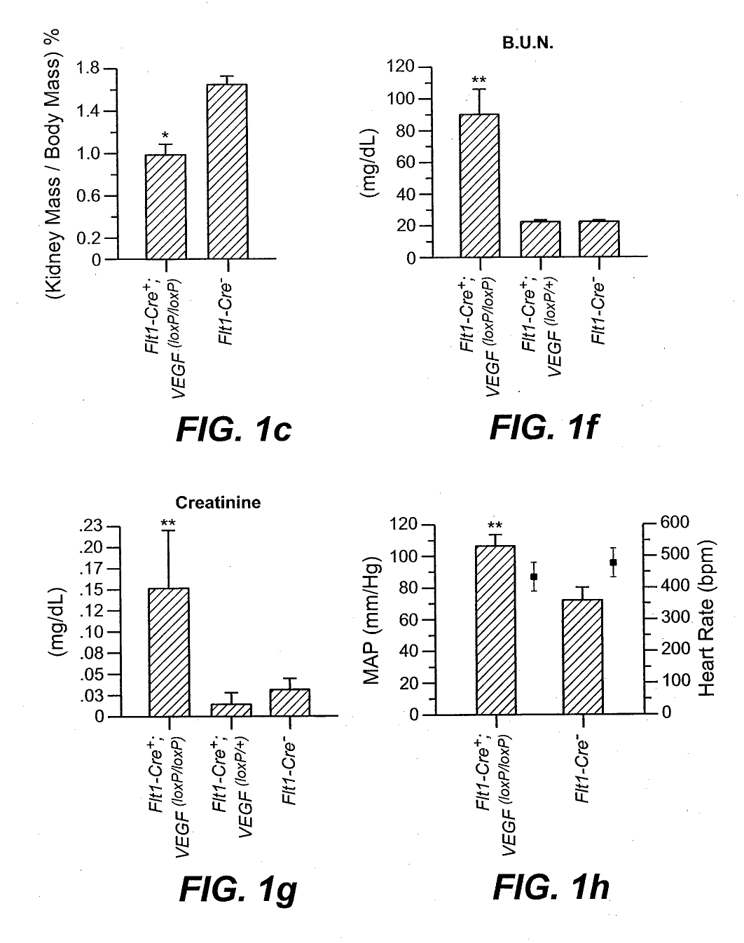

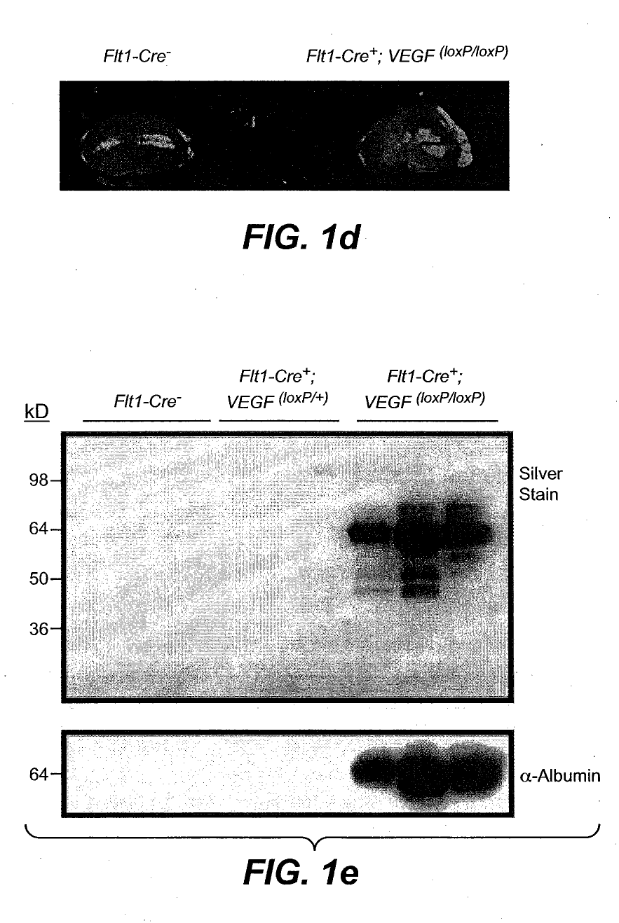

Flt-CRE⁺; VEGF^{(loxP/loxP)} mice were born at the expected Mendelian ratios (FIG. 1, Panel a), however, decreased survival was evident from 4 weeks of age on, with greater than 95% of Flt-CRE⁺; VEGF^{(loxP/loxP)} mice dead by 12 weeks (FIG. 1, Panel b). Closer inspection revealed that the Flt-CRE⁺; VEGF^{(loxP/loxP)} mice lacked a spleen and kidney mass was significantly reduced. See FIG. 1, Panel c. There was also reduced kidney vascularization and appearance of cystic kidney lesions, indicative of bilateral kidney disease. See, FIG. 1, Panel d. Other organs with LacZ positive vasculature such as lung, liver, heart, brain and skeletal muscle, did not display any significant changes in morphology, weights or vascularization.

Urine analysis revealed proteinuria exceeding 500 mg/dL in 4 to 5 week old Flt-CRE⁺; VEGF^{(loxP/loxP)} mice. Silver staining and western blotting analysis revealed massive amounts of albumin, indicative of defective glomerular filtration barrier functions, in the urine of Flt-CRE⁺; VEGF^{(loxP/loxP)}, but not Flt-CRE⁺; VEGF^{(loxP/-)} or Flt1-Cre− mice. See, FIG. 1, Panel e. Blood chemistry analysis revealed a 4-fold increase in levels of blood urea nitrogen (B.U.N.) and serum creatinine in the Flt-CRE⁺; VEGF^{(loxP/loxP)} mice compared with control mice (see FIG. 1, Panels f and g), whereas serum levels of sodium, potassium, chloride and calcium were unaffected. Consistent with the pathology associated with renal failure, blood pressure was significantly elevated in Flt-CRE⁺; VEGF^{(loxP/loxP)} transgenic mice relative to Flt-CRE⁻ control littermates. See, FIG. 1, Panel h. Combined, these can indicate that Flt-CRE⁺; VEGF^{(loxP/loxP)} mice progressively develop kidney malfunction associated with proteinuria and hypertension, culminating in end-stage renal failure.

### Flt1-Cre Transgene Expression and VEGF-A Gene Ablation in Kidney Mesangial Cells

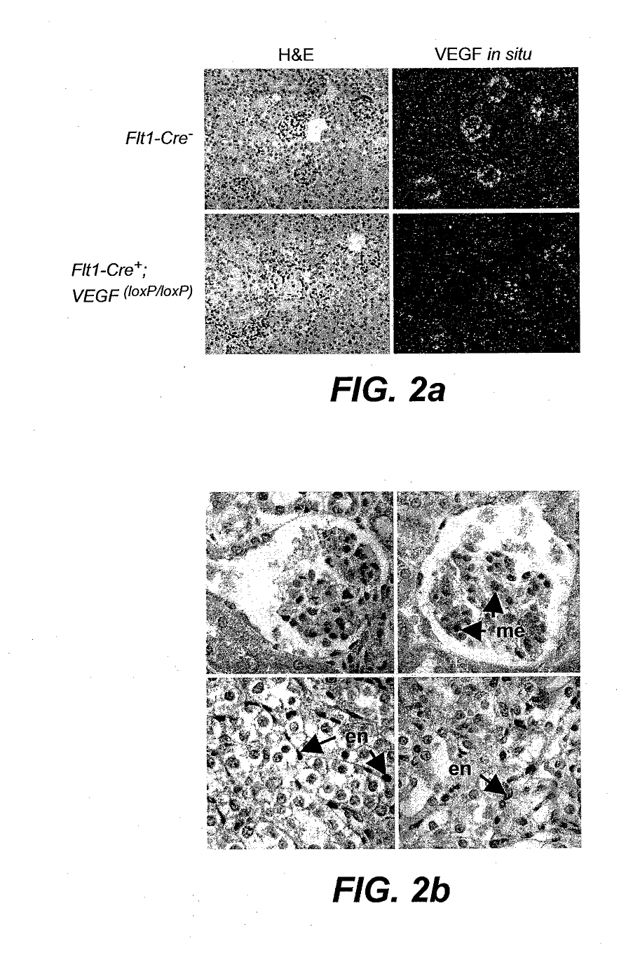

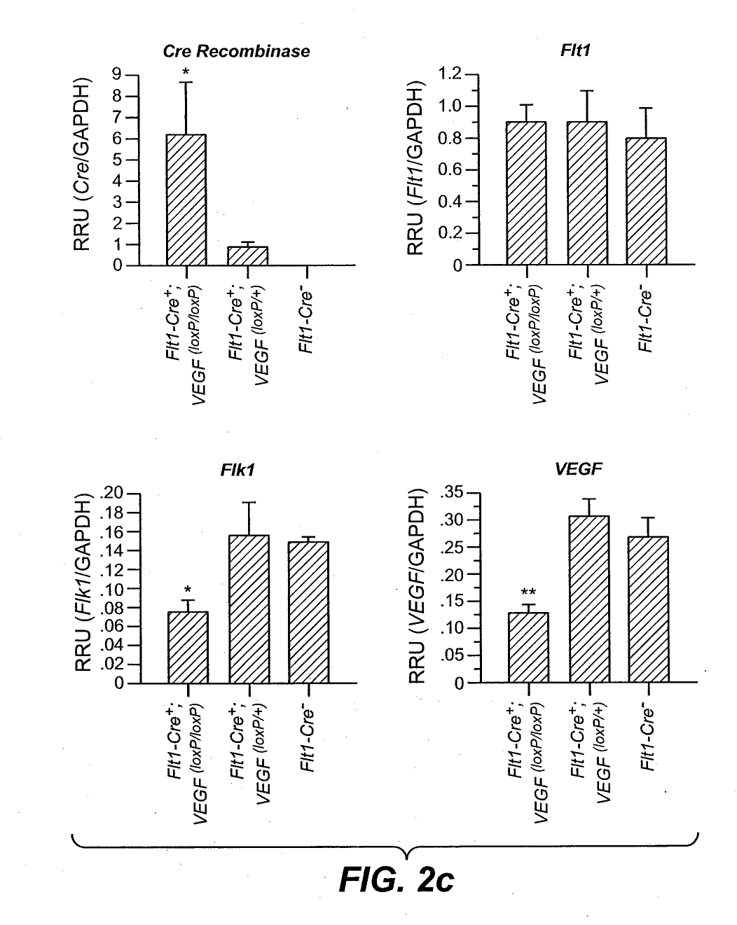

Using immunhistological approaches, we co-localized VEGF-A expression and podocytes expressing α-Wilms Tumor nuclear protein (see, e.g., Haas, C., et al. MHC antigens in interferon gamma (IFN gamma) receptor deficient mice: IFN gamma-dependent up-regulation of MHC class II in renal tubules. Kidney-Int 48:1721-7 issn: 0085-2538 (1995)) in glomeruli of 4 weeks old Flt1-Cre⁺; VEGF(loxP/loxP) mice, highlighting that the majority of VEGF-A expression was confined to podocytes. VEGF-A was detected in glomerular podocytes and mesangial cells when frozen sections of kidneys of Flt1-Cre⁺; VEGF^{(loxP/loxP)}; ROSA26⁺ mice aged 4-weeks were stained with antibodies to detect mesangial cells (anti-Integrin α8), endothelial cells (anti-CD31) and podocytes (anti-WT-1) along with co-staining of the sections with α-VEGF to identify the glomerular cell types that express VEGF-A. Merged images indicated VEGF-A expression is detectable in WT-1-positive podocytes and significant, but lower VEGF-A expression is detectable in glomerular mesangial cells. In situ hybridization of VEGF-A confirmed high levels of VEGF-A expression in podocytes, as shown by the abundance of silver grains at the periphery of the Flt1-Cre− glomeruli of 7 week-old mice. See FIG. 2, Panel a. Co-staining of integrin α8 positive mesangial cells (see, e.g., Hartner, A., et al., Alpha8 integrin in glomerular mesangial cells and in experimental glomerulonephritis. Kidney Int 56:1468-80 (1999)) with VEGF-A revealed weaker, but significant expression in mesangial cells in 4 week old mice. See FIG. 2, Panel a. However, we were unable to detect VEGF-A/CD31 double positive endothelial cells in any of the kidney sections analyzed, consistent with the absence of VEGF-A in glomerular endothelial cells described previously. See, e.g., Simon, M. et al. Expression of vascular endothelial growth factor and its receptors in human renal ontogenesis and in adult kidney. Am-J-Physiol 268:F240-50 issn: 0002-9513 (1995); and, Noguchi, K. et al. Activated mesangial cells produce vascular permeability factor in early-stage mesangial proliferative glomerulonephritis. J Am Soc Nephrol 9:1815-25 (1998).

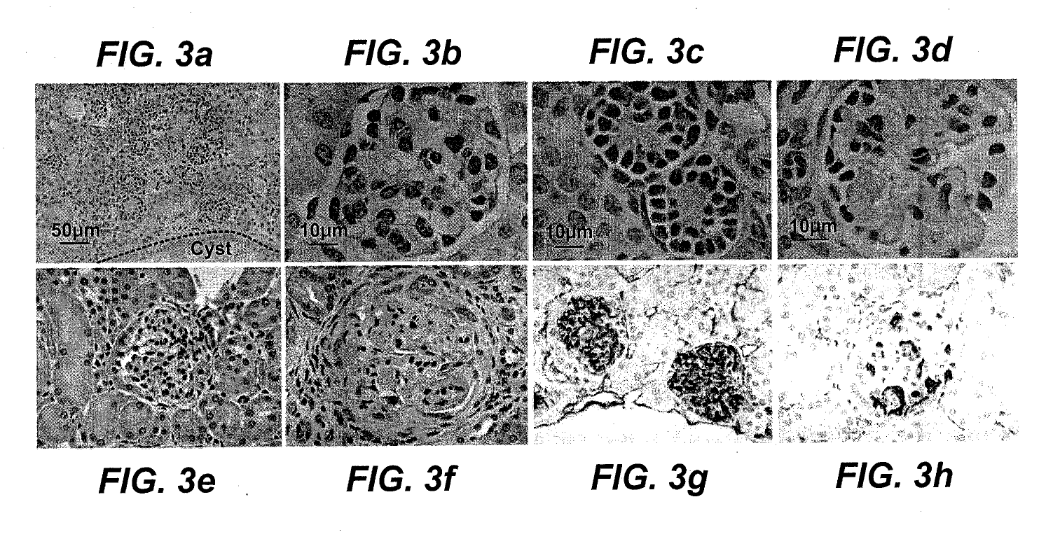

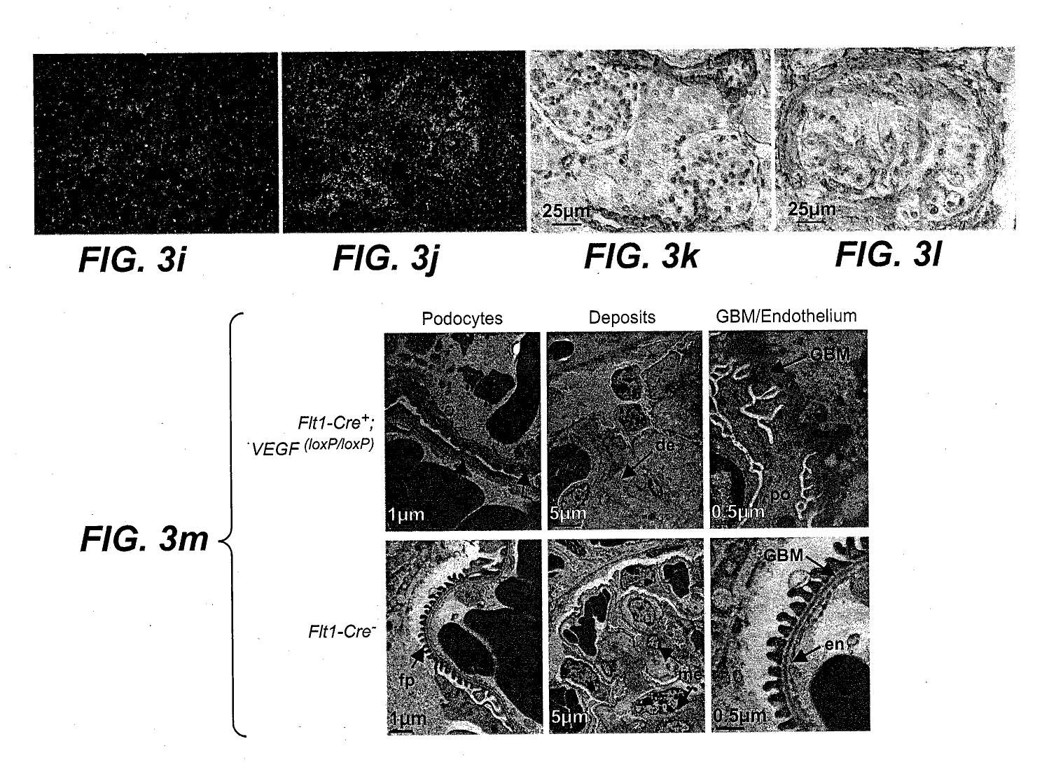

To determine the glomerular cell-types expressing Cre-recombinase during embryonic and postnatal development, immunofluorescent histochemistry was employed using an antibody detecting β-galactosidase (anti-beta-Gal). Analysis of kidney sections of 4 week old Flt1-Cre+; VEGF(loxP/loxP) mice revealed increased numbers and staining of β-Gal/integrin α8 double-positive mesangial cells in Flt1-Cre+; VEGF(loxP/loxP) compared with Flt1-Cre⁺; VEGF(loxP/+) littermates. However, we did not detect cells double positive for β-Gal and the podocyte marker WT-1 or the endothelial cell marker CD31. Immunohistochemistry revealed that the Flt1-Cre transgene was detected in mesangial cells, but not glomerular endothelial cells, whereas tubular endothelial cells, known to express Flt-1, were positive. See FIG. 2, Panel b. Genomic PCR revealed significant levels of VEGF ablation in kidneys during embryonic development (E17.5 and E18.5), and in mesangial cell explants from kidneys of one week old Flt1-Cre+; VEGF(loxP/wt) mice following expansion for 7 to 10 days. These findings are consistent with a previous report (see, e.g., Takahashi, T. et al. Protein tyrosine kinases expressed in glomeruli and cultured glomerular cells: Flt-1 and VEGF expression in renal mesangial cells. Biochem Biophys Res Commun 209:218-26 (1995)), suggesting that mesangial cells within the kidney glomerulus can express both, VEGF-A and Flt1 and further support the notion, that VEGF-gene ablation in glomeruli of Flt1-Cre+; VEGF(loxP/loxP) mice occurs in at least the mesangial cells. VEGF-A gene ablation may occur in other glomerular cells or in cells outside the glomerular compartment, which are not detected in our assays, and may indirectly contribute to the glomerular changes observed. Quantitative gene-expression analysis of RNA confirmed that the tissue damage and ongoing repair processes in Flt1-Cre+; VEGF(loxP/loxP) kidney causes marked upregulation of Cre-recombinase (see FIG. 2, Panel c). Concomitantly, a down regulation of Flk1 (VEGFR-2) and VEGF-A was detected (FIG. 2, Panel c), consistent with VEGF-A gene ablation and a reduction in glomerular vascularity (FIG. 3, Panel g, and h), while Flt-1 levels remained unchanged. In situ hybridization experiments with of Flt1-Cre+; VEGF(loxP/loxP) kidneys revealed markedly reduced VEGF-A expression in all glomerular cell types by 7 weeks of age. Immunohistochemical analysis of one-week old kidneys for VEGF and B-Gal expression identified 3 major classes of glomeruli: 1.) glomeruli expressing normal levels of VEGF in the absence of β-Gal staining. 2.) glomeruli displaying reduced VEGF and punctate β-Gal expression and 3.) undetectable VEGF levels in presence of high β-Gal staining. VEGF gene ablation in kidney mesangial cells may occur throughout postnatal development and may be associated with decreased podocyte VEGF expression and/or podocyte cell death. In these experiments, the findings identify VEGF expression by podocytes as a downstream target of the autocrine regulatory loop by VEGF in kidney mesangial cells.

Hypoxia was found to upregulate expression of both VEGF-A and VEGFR-1 (see, e.g., Gerber, H. P., et al. Differential transcriptional regulation of the two vascular endothelial growth factor receptor genes. Flt-1, but not Flk-1/KDR, is up-regulated by hypoxia. J Biol Chem 272:23659-67 (1997)) and increased renal hypoxia was reported of glomerulonephritis. See, e.g., Nangaku, M. Mechanisms of tubulointerstitial injury in the kidney: final common pathways to end-stage renal failure. Intern Med 43:9-17 (2004). When analyzing kidney glomeruli of 4 week old Flt1-Cre+; VEGF(loxP/loxP) for hypoxic regions by using (Hypoxyprobe™-Mab1), increased hypoxia was consistently found in conditional knock-out mice compared to wild-type littermates, indicating that hypoxia may accelerate VEGF gene ablation via upregulation of Flt-CRE. Regional variations in hypoxia combined with variations in the severity of disease progression within individual glomeruli may explain the frequently focal expression of the Flt1-Cre transgene and the lack of transgene expression in glomerular endothelial cells, which are in direct contact with the normoxic circulation.

### Pathophysiological Changes in Glomeruli of Flt-CRE⁺; VEGF^{loxP/loxP)} Transgenic Mice

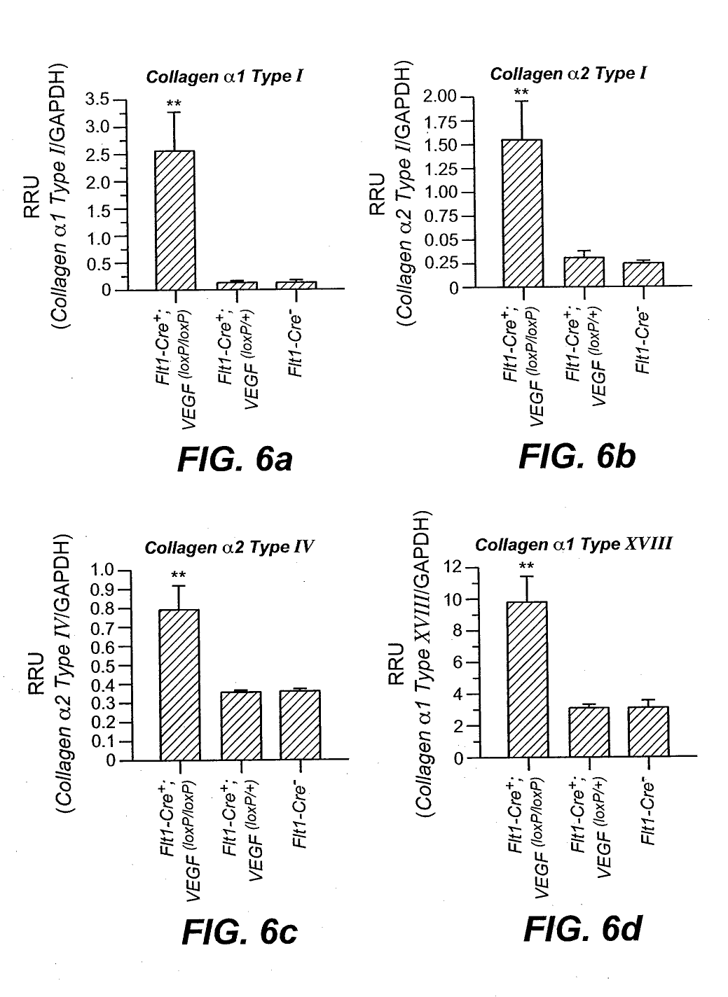

The histopathologic changes occurring in Flt-Cre+; VEGF(loxP/loxP) mice were analyzed to understand the cellular events leading to kidney failure. Abnormal glomerular structures were evident in Flt-Cre+; VEGF(loxP/loxP) mice examined by light microscopy on weeks 2, 3, and 7. In mice aged 2 weeks, cysts were present throughout the renal cortex (FIG. 3, Panel a) and 2 types of glomerular structures could be discerned that were distinct in appearance to the glomeruli of their age-matched WT littermates (FIG. 3, Panel b). In 2 week old Flt-Cre+; VEGF(loxP/loxP) mice, underdeveloped glomeruli consisting of podocytes surrounding an acellular core were frequently observed (FIG. 3, Panels a and c). Glomeruli with this appearance may fail to function, due to an absence of capillary loops, and that they may fail to develop beyond this stage, as there is no evidence of a similar structure in the kidney of mice aged 7 weeks (FIG. 3, Panels e and f). Numerous glomeruli within the kidney of 2-3 week old Flt-Cre+; VEGF(loxP/loxP) mice were markedly enlarged and displayed abundant eosinophilic, proteinaceous depositions throughout the glomeruli and absence or collapse of existing capillary loops (FIG. 3, Panel d). These glomeruli have a similar appearance to typical glomeruli of Flt-Cre+; VEGF(loxP/loxP) mice aged 7 weeks, which display glomerulosclerosis, fibrosis, and focal interstitial nephritis (FIG. 3, Panel f). Within each kidney, individual glomeruli are affected to different degrees, with mild to severe changes evident. In addition, large cysts lined with transitional epithelium (FIG. 3, Panel a), and scattered groups of dilated cortical tubules filled with proteinaceous material were observed (compare FIG. 3, Panels e and f). Decreased cellularity was evident throughout the glomeruli of Flt-Cre+; VEGF(loxP/loxP) mice when compared with that observed in WT kidney, which can be attributed, in part, to reduced numbers of endothelial cells (compare FIG. 3, Panels g and h). Decreased CD31 staining in the Flt-Cre+; VEGF(loxP/loxP) kidneys was accompanied by extensive laminin and focal collagen IV depositions in many sclerotic glomeruli, as determined by RT-PCR (FIG. 6, a-d) and immunohistochemical staining. For example, in kidnety section of 7-week old mice, laminin deposition was detected in the tubules and was throughout the glomeruli of the diseased kidneys, and increased collagen IV staining was detected in diseased tissue compared to the wild-type littermate. Furthermore, transforming growth factor-β (tgf-β, FIG. 3, Panels i and j), a mediator of glomerular fibrosis and tissue damage frequently upregulated in kidney disease (reviewed by Schnaper, H. W., et al. TGF-beta signal transduction and mesangial cell fibrogenesis. Am J Physiol Renal Physiol 284:F243-52 (2003)), as well as alpha smooth muscle actin (FIG. 3, Panels k and l), a marker for activated mesangial cells, were both markedly elevated in damaged kidneys.

Ultrastructural analysis of kidney sections by transmission electron microscopy identified defects in the mesangium and other features consistent with focal glomerulosclerosis including podocyte foot process fusion and expansion of mesangial matrix in Flt-CRE⁺; VEGF^{(loxP/loxP)} kidneys (FIG. 3, Panel m). Although the endothelium appears healthy in some glomeruli, loss of fenestrations and massive expansion of the glomerular basement membrane is observed in glomeruli with more advanced lesions. In addition, loss of mesangial cells and electron dense deposits was present (FIG. 3, Panel m) in underdeveloped glomeruli.

VEGF Gene Ablation in Glomerular Mesangial Cells is Associated with Immunoglobulin M (IgM) Deposits and Complement Activation.

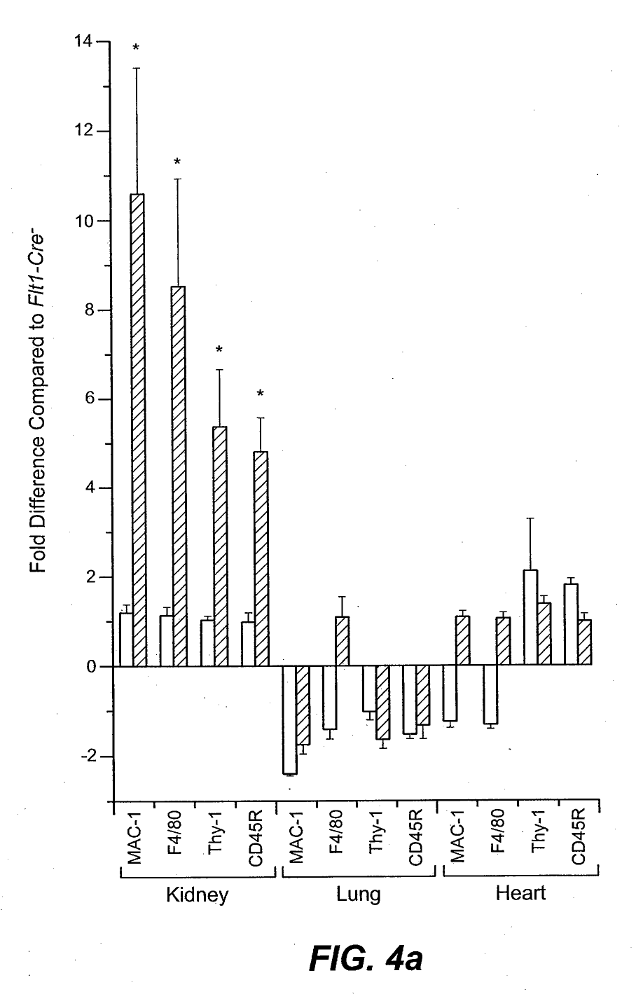

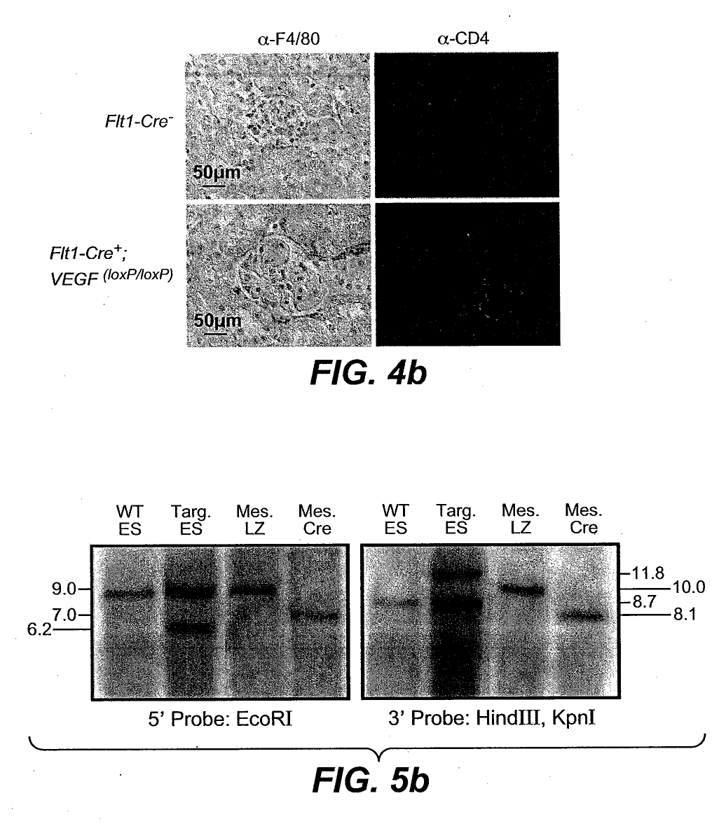

Evidence of a heightened immune response in the kidneys of Flt-CRE⁺; VEGF^{(loxP/loxP)} mice was suggested by elevations in circulating lymphocytes. Real time quantitative RT-PCR analysis of kidney RNA for markers of the monocyte/macrophage, B-cell and T-cell lineages (CD11b, F4/80, CD45R and Thy-1, respectively) detected immune infiltrates in kidneys of Flt-CRE⁺; VEGF^{(loxP/loxP)} mice, but not in control littermates (FIG. 4, Panel a). Immunohistochemical analysis revealed increased numbers of cells expressing F4/80 and CD4, a marker of a subset of T-cells, in the kidneys of Flt-Cre+; VEGF(loxP/loxP) mice. See FIG. 4, Panel b. Immune cell infiltrations appeared to be specific for kidney tissues, as these cell lineage markers were not elevated in the lungs or hearts of Flt-Cre+; VEGF(loxP/loxP) transgenic mice.

Among the Ig subclasses of antibodies analyzed, IgM, but not IgG, IgA, IgD, or IgE was found deposited in diseased glomeruli, e.g, by immunofluorescent staining of kidney cortical tissue from Flt1-Cre⁺; VEGF^{(loxP/loxP)} mice aged 5 weeks using monoclonal antibodies specific for IgM and each murine isotype of IgG. Furthermore, a marked increase in C1q, C3, and C4 proteins of the complement pathway was detected (e.g., by immunofluorescence employing monoclonal antibodies specific for C1q, C3, and C4 components of the pathway), particularly in the glomeruli of Flt-Cre+; VEGF(loxP/loxP) kidney compared with WT kidney. Increased C1q, C3, and C4 were detected in mice aged 1 week, suggesting that complement-mediated cell lysis may significantly contribute to the kidney damage in Flt-Cre+; VEGF(loxP/loxP) mice. Evidence of complement-mediated damage in the kidneys of mice with haplo-insufficient podocyte-selective deletion of VEGF was not detected.

### VEGF Gene Ablation In Vitro Adversely Affects Mesangial Cell Survival: Evidence of VEGF Acting Via an Internal Autocrine Loop in Mesangial Cells.

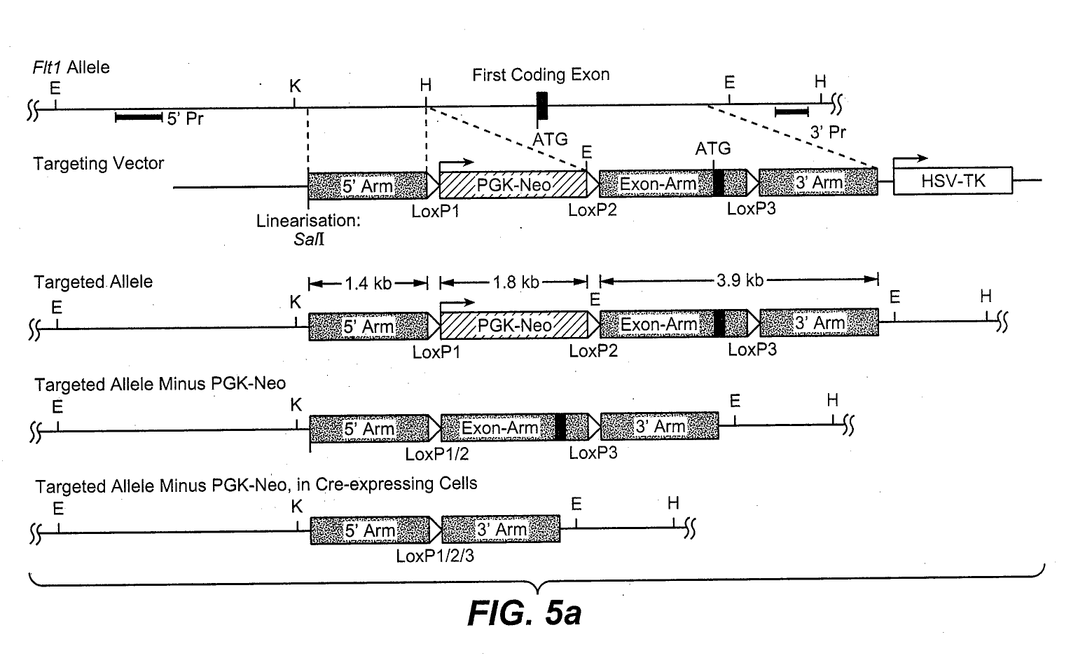

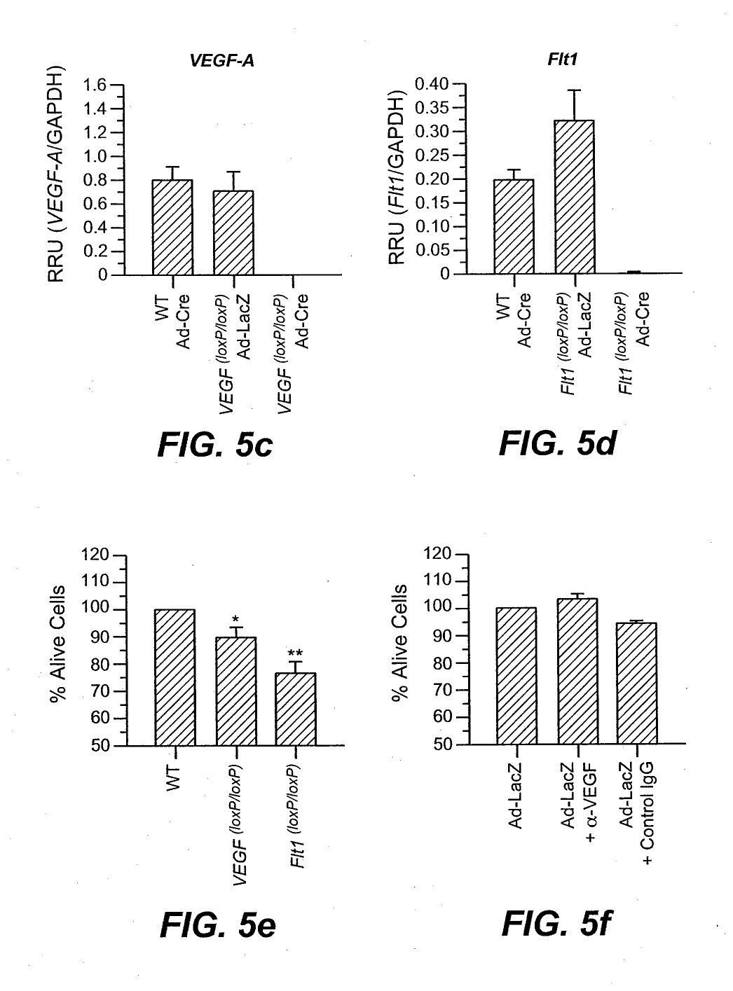

To investigate the effects of VEGF-A and Flt1 gene ablation on mesangial cell grown in vitro, we generated mice with a conditional alleles for the Flt1 allele (Flt1-lox/loxP). See FIG. 5, Panel a. A targeting vector in which exon 1 of the mouse Flt1 gene is flanked by loxP sites was generated and used for homologous recombination in mouse embryonic stem cells. Flt1(loxP/loxP) mice were born at the expected Mendelian frequencies, indicating that the presence of 2 loxP sites did not interfere with mouse development. Homogeneous preparations of mesangial cells from WT VEGF(loxP/loxP) and Flt1(loxP/loxP) mice were obtained from glomerular isolates and infected with either control adenovirus expressing LacZ (Ad-LacZ) or adenovirus expressing Cre-recombinase (Ad-Cre). Flt1 and VEGF-A gene ablation frequencies in vitro were monitored by Southern blot analysis (FIG. 5, Panel b) and real time RT-PCR (FIG. 5, Panels c and d) and found to be >95%. Flt-1 or VEGF-A gene ablation in mesangial cells caused a significant reduction in cell survival (FIG. 5, Panel e), indicating that VEGF regulates mesangial cell survival in a cell autonomous manner, mediated by Flt1. Addition of a neutralizing VEGF antibody (α-VEGF, G6-23) did not impact on mesangial cell survival (FIG. 5, Panel f). As G6-23 is excluded from the intracellular compartment, the failure to recapitulate the decrease in mesangial cell survival observed in VEGF-A- or Flt1-deficient mesangial cells indicates that VEGF-A may act via an internal autocrine loop. The reduction in survival of Flt1-deficient mesangial cells is greater than that caused by VEGF-A deficiency alone, suggesting that other ligands for Flt1, such as PlGF or VEGF-B, may also contribute.

VEGF Gene Ablation in Mesangial Cells Induces Changes in Gene Expression Consistent with Increased ECM Production.

We conducted a gene ontology (GO) analysis of genes that are differentially expressed in VEGF-A-deficient mesangial cells. When selected for changes greater than 2-fold (absolute value) and p-values of <0.05 (Students' t-test), we found 480 out of 11810 genes analyzed to be significantly upregulated in VEGF-A-deficient mesangial cells when compared with WT mesangial cells. Comparison of the GO annotations between the class of upregulated genes and a control set of all genes revealed significant differences in genes involved in regulating chemotaxis, structural integrity or ECM production, cell migration and the humoral defense mechanism were significantly over-represented (Table 2). We performed an identical analysis for genes downregulated in VEGF-A deficient mesangial cells and identified six categories that were significantly over-represented. Three of the six categories belonged to the super-class of proteolytic genes and one included genes involved in cell communication (Table 2). Signal pathway analysis performed using the Ingenuity Pathways system further revealed that the expression of genes belonging to the subclasses defined as cellular growth and proliferation, cell assembly, organization and compromise pathway were significantly altered in VEGF-deficient mesangial cells (Table 2). We also identified that the expression of 46% to 57% of the genes belonging to the categories of molecular networks involved in protein synthesis, cell death, cell-cell signaling and immune response were significantly affected by VEGF deficiency (Table 2). Candidate genes associated with mesangial matrix accumulation or glomerular disease were also significantly dysregulated in VEGF-A-deficient mesangial cells. Among them, tgf-β1 (see, e.g., Schnaper, H. W., et al. TGF-beta signal transduction and mesangial cell fibrogenesis. Am J Physiol Renal Physiol 284:F243-252 (2003)), angiopoietin-1 (see, e.g., Satchell, S. C., and Mathieson, P. W. Angiopoietins: microvascular modulators with potential roles in glomerular pathophysiology. J Nephrol 16:168-178 (2003)), and COX-2 (reviewed in (Nasrallah, R. & Hebert, R. L. Prostacyclin signaling in the kidney: implications for health and disease. Am J Physiol Renal Physiol 289:F235-46 (2005)) were upregulated, whereas platelet-derived growth factor receptor-β (Pdgfr-β) and Pdgf-c (reviewed in (Betsholtz, C. et al. Role of platelet-derived growth factor in mesangium development and vasculopathies: lessons from platelet-derived growth factor and platelet-derived growth factor receptor mutations in mice. Curr Opin Nephrol Hypertens 13:45-52 (2004))) were down-regulated. The changes in gene expression in VEGF-A-deficient mesangial cells in vitro highlight a shift toward accumulation of ECM mesangium components and identified VEGF-A to regulate these processes in a cell-autonomous manner.

Table 2. Changes in Gene Expression Associated with VEGF-Deficiency in Mesangial Cells.

Gene ontology classes that were significantly over-represented in those genes either up- or down-regulated in VEGF-A deficient mesangial cells.

### Gene Ontology Families UP-REGULATED IN VEGF-deficient Mesangial Cells

| Ontology | FOLD-ENRICHMENT | P-VALUE |
|---|---|---|
| Structural Molecular Activity | 2.1 | p = 0.006 |
| Chemotaxis | 3.2 | p = 0.006 |
| Regulation in Cell Migration | 7.0 | p = 0.001 |
| Humoral Defense Mechanism | 8.9 | p = 0.003 |

### Gene Ontology Families DOWN-REGULATED in VEGF-deficient Mesangial Cells

| Ontology | FOLD-ENRICHMENT | P-VALUE |
|---|---|---|
| Serine-type endopeptidase | 6.6 | p = 0.0001 |
| activity | | |
| Chymotrypsin activity | 6.5 | p = 0.0001 |
| Trypsin activity | 6.3 | .p = 0.002 |
| Cell Communication | 11.3 | .p = 0.008 |

Ingenuity Pathways Analysis identified functional pathways represented by genes whose expression was significantly differentially regulated in VEGF-A deficient mesangial cells.
Genes Classified by Functional Pathways that are Significantly Altered in Expression in VEGF-Deficient Mesangial Cells

| | | | % of Genes |
|---|---|---|---|
| | | Genes DOWN- | in network |
| Gene Pathway/Function | Genes UP-Regulated | Regulated | represented |
| Cellular Growth and | ADD3, APPBP2, | GPX1, HBEGF, | 100% |
| Proliferation, Cellular | ARF6, ARID4A, | HSPA1B, LAMP1, | |
| Assembly and | CEACAM1, FCGR1A, | LGALS1, MFGE8, | |
| Organisation, Cellular | FRAP1, ING1, MC3R, | PIK3R2, PPP1R15A, | |
| Compromise | NCL, NR3C1, PIK3R1, | RHOD, RPS6, | |
| | PLK4, PRAP1, PTPRF, | SERPINE1, | |
| | SDC2, TGFBR1, | SMARCA4, | |
| | VAV3 | SMARCB1, | |
| | | TMSB10, | |
| | | TNFRSF1A, | |
| | | TRIM28, VEGF | |
| Protein Synthesis, Post- | UBE2V2 | ACTN4, FAU, | 57% |
| Translational | | RPL12, RPL22, | |
| Modification, Cancer | | RPL26, RPL29, | |
| | | RPL18A, RPL23A, | |
| | | RPL37A, RPLP2, | |
| | | RPS2, RPS3, RPS13, | |
| | | RPS26, RPS28 | |
| Post-Translational | ABCC3, CP, DBT, | ATP5H, MAFF, | 49% |
| Modification, Cell Death, | FCGR1A, H2-D1, | MYH9, NUTF2, | |
| Immunological Disease | KIF3C | P4HB, PP1B, RPS11, | |
| | | SLC7A1, ST3GAL3, | |
| | | TNK1, TPD52L2 | |
| Cell-To-Cell Signaling | GAS7, ITGB4BP, | E1F2S2, FCGR3A, | 49% |
| and Interaction, | RIN2, STXPB5 | GNB2L1, PDGFC, | |
| Hematological System | | PDGFRB, PFKP, | |
| Development and | | PTPN11, RAPGEF1, | |
| Function, Immune | | SLC9A3R1, SRP14, | |
| Response | | TBC1D10A, | |
| | | YWHAB, YWHAQ | |
| Cell Death, Gene | EFNA4, ING1, PARC, | CKAP4, FLII, GPI, | 46% |
| Expression, Cell Cycle | PEX6, SESN1, | HBEGF, HSPH1, | |
| | TCF7L2, UBE2D3 | PAXIP1L, PHC2, | |
| | | SSRP1, TADA3L | |

Renal Pathologies Associated with Decreased VEGF Expression During Kidney Development

A cell autonomous function of VEGF-A in kidney mesangial cells, mediated by Flt-1, is identified and a role of this regulatory mechanism during kidney development is described. Evidence is provided that interference with this regulatory mechanism can cause some of the pathophysiologic features associated with glomerulosclerosis. The progressive renal failure in this model represents a developmental injury model, rather than an adult onset model of glomerulonephritis. There are some similarities in the kidney pathology in this model and human kidney diseases associated with lower VEGF levels in the kidneys (see, e.g., Schrijvers, B. F., et al., The role of vascular endothelial growth factor (VEGF) in renal pathophysiology. Kidney Int 65:2003-17 (2004)), e.g., in glomerular injury, sclerosis, and inflammatory deposits and complement activation. See, e.g., Isselbacher, K. J. et al. Harrison's Principles of Internal Medicine, (1994) (McGraw-Hill Inc., New York). The Flt-Cre+; VEGF(loxP/loxP) mouse is a genetic model displaying accumulation of IgM deposits and activation of C1q, C3, and C4 in diseased kidneys. Our findings are different from previous observations in mice aged 9-12 weeks with podocyte-specific VEGF-A haplo-deficiency, which developed end-stage renal failure in the absence of immune complex formation. See, e.g., Eremina, V. et al. Glomerular-specific alterations of VEGF-A expression lead to distinct congenital and acquired renal diseases. J Clin Invest 111:707-16 (2003).

The majority of reports investigating VEGFR1 and/or VEGF-A expression in primary cell isolates and in healthy and diseased kidneys, identified mesangial cells as most likely to co-express both genes (see, e.g., Thomas, S. et al. Vascular endothelial growth factor receptors in human mesangium in vitro and in glomerular disease. J Am Soc Nephrol 11: 1236-43 (2000); Gruden, G. et al. Mechanical stretch induces vascular permeability factor in human mesangial cells: mechanisms of signal transduction. Proc Natl Acad Sci USA 94:12112-6 (1997); Harper, S. J. et al. Expression of neuropilin-1 by human glomerular epithelial cells in vitro and in vivo. Clin Sci (Lond) 101:439-46 (2001); Takahashi, T. et al. Protein tyrosine kinases expressed in glomeruli and cultured glomerular cells: Flt-1 and VEGF expression in renal mesangial cells. Biochem Biophys Res Commun 209:218-26 (1995); Simon, M. et al. Expression of vascular endothelial growth factor and its receptors in human renal ontogenesis and in adult kidney. Am-J-Physiol 268 (1995); and, Noguchi, K. et al. Activated mesangial cells produce vascular permeability factor in early-stage mesangial proliferative glomerulonephritis. J Am Soc Nephrol 9:1815-25 (1998)), rather than podocytes, which are known to express highest levels of VEGF-A (see, e.g., Berse, B., et al. Vascular permeability factor (vascular endothelial growth factor) gene is expressed differentially in normal tissues, macrophages, and tumors. Molecular Biology of the Cell 3:211-20 (1992); and, Brown, L. F. et al. Expression of vascular permeability factor (vascular endothelial growth factor) by epidermal keratinocytes during wound healing, Journal of Experimental Medicine 176:1375-9 (1992)), or endothelial cells expressing VEGFR-1 (see, e.g., Thomas, S. et al. Vascular endothelial growth factor receptors in human mesangium in vitro and in glomerular disease. J Am Soc Nephrol 11:1236-43 (2000)). Cre-positive podocytes were not detected in Flt1-Cre+, VEGF(LoxP/LoxP), ROSA26 compound transgenic mice at various stages during development. The absence of VEGFR-1/2 expression in non-transformed podocytes is consistent with previous reports (see, e.g., Gruden, G. et al. Mechanical stretch induces vascular permeability factor in human mesangial cells: mechanisms of signal transduction. Proc Natl Acad Sci USA 94:12112-6 (1997); Harper, S. J. et al. Expression of neuropilin-1 by human glomerular epithelial cells in vitro and in vivo. Clin Sci (Lond) 101:439-46 (2001); and, Takahashi, T. et al. Protein tyrosine kinases expressed in glomeruli and cultured glomerular cells: Flt-1 and VEGF expression in renal mesangial cells. Biochem Biophys Res Commun 209:218-26 (1995)) and our own observations. However, transformation of primary murine podocytes with SV40 large T-antigen resulted in VEGFR-1 and VEGF-A expression in some transformed podocyte cell lines. See, e.g., Chen, S. et al. Podocyte-derived vascular endothelial growth factor mediates the stimulation of alpha3(IV) collagen production by transforming growth factor-beta1 in mouse podocytes. Diabetes 53:2939-49 (2004).

Evidence is provided for the existence of two regulatory mechanisms in the development of the kidney glomeruli controlled by VEGF: 1.) the paracrine mechanism between podocyte and glomerular capillary endothelial cells, which was suggested to be involved in the induction and maintenance of fenestrae and/or glomerular filtration rates (reviewed in Schrijvers, B. F., et al. The role of vascular endothelial growth factor (VEGF) in renal pathophysiology. Kidney Int 65:2003-17 (2004)); and, 2.) the autocrine loop in mesangial cells regulating ECM production and podocyte functions, including VEGF expression. The model, wherein VEGF production in podocytes is a downstream function of VEGF's autocrine role in mesangial cells, can account for the overlap in the phenotypes in mice with VEGF-deficiency in either cell-type. Mesangial VEGF-A deficiency also led to an additional set of renal changes not found in podocyte-specific, VEGF-haplo-insufficient mice, including the presence of infiltrating inflammatory cells (FIG. 4 a-b) and the expansion of the glomerular basement membrane (FIG. 3m). These findings suggest that the cell-type affected, in addition to the overall reduction in VEGF-A expression, may represent a key determinant for kidney pathology. Administration of compounds stimulating VEGF signaling, in particular VEGFR-1, may be beneficial for the treatment of a subset kidney diseases associated with glomerular sclerosis.

### Differential Effects Between Genetic and Biochemical Inactivation of VEGF on Mesangial Cells: Further Evidence for Internal, Autocrine VEGF-A Effector Functions as a Regulatory Mechanism

A prerequisite for the detection of the cell autonomous function of VEGF in mesangial cells was the apparent failure of podocyte-derived VEGF-A to rescue VEGF-A-deficient mesangial cells via para- or juxtacrine signaling. Without being limited to a single model, the identification of podocyte VEGF expression to be a downstream function of VEGF's autocrine loop in mesangial cells provides a rational for the inability of podocytes to rescue VEGF-A deficient mesangial cells. A cell autonomous function for VEGF-A in the regulation of hematopoietic stem cell survival (HSCs) was previously reported. See, e.g., Gerber, H. P. et al. VEGF regulates haematopoietic stem cell survival by an internal autocrine loop mechanism. Nature 417, 954-8 (2002). In analogy, administration of an antibody neutralizing VEGF-A (G6-23) to mesangial cell grown in culture did not impact on mesangial cell numbers, in contrast to cells deficient for VEGF-A or Flt1. Without being limited to one theory, the findings suggest that autocrine signaling may represent a more general regulatory mechanism to separate VEGF's paracrine functions controlling blood vessel formation from non-angiogenic effector functions (reviewed in e.g., Gerber, H. P. & Ferrara, N. The role of VEGF in normal and neoplastic hematopoiesis. J Mol Med 81:20-31 (2003)) in multiple cell types.

### Example 2

### VEGFR Selective Variants of VEGF

Generation and characterization of VEGF variants that selectively bind and activate a specific VEGF receptor (such as KDR or Flt-1) have been known in the art and described in, for example, Li et al. J. Biol. Chem. 275:29823 (2000); Gille et al. J. Biol. Chem. 276:3222-3230 (2001); PCT publications WO 00/63380 and 97/08313; and U.S. Pat. No. 6,057,428, the disclosure of which are expressly incorporated herein by reference.

Specifically, a VEGF variant with high selectivity for the Flt-1 receptor was generated by combining four mutations that greatly affected KDR but not Flt-1 binding. Mutation of Ile 43, Ile 46, Gln 79 and/or Ile 83 showed that the side chains of these residues are critical for tight binding to KDR but unimportant for Flt-1-binding. Li et al. (2000) supra. A Flt-sel variant was constructed with alanine substitutions at positions Ile 43, Ile 46, Gln 79 and Be 83, using site directed mutagenesis methods described by Kunkel et al. Methods Enzymol. 204:125-139 (1991). This particular Flt-sel variant can also be represented by the identifier, I43A/I46A/Q79A/I83A. The corresponding codons for these four alanine substitutions at positions 43, 46, 79 and 83 are GCC/GCC/GCG/GCC, respectively.

Various assays were conducted to examine the properties and biological activities of the I43A/I46A/Q79A/I83A Flt-sel variant. Li et al. (2000) supra. For example, quantitative binding measurements were carried out using a soluble radio-immuno receptor-binding assay (RIA). In the assay, native VEGF(8-109) had affinities for KDR and Flt-1 of 0.5 nM and 0.4 nM, respectively. Flt1-sel was found to have at least 470-fold reduced KDR-binding affinity in this assay. Small reductions in Flt-1-binding had been observed from the individual point mutants in the ELISA, the Flt-sel variant's affinity for Flt-1 was essentially identical to that of the native protein.

The specification is considered to be sufficient to enable one skilled in the art to practice the invention. It is understood that the examples and embodiments described herein are for illustrative purposes only. Indeed, various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

We claim:

1. A method of treating a renal disease, the method comprising:
administering an effective amount of a VEGFR modulating agent to a subject with the renal disease,
wherein the VEGFR modulating agent comprises a Flt1 agonist.

2. The method of claim 1, wherein the Flt1 agonist is a Flt1 agonist antibody.

3. The method of claim 1, wherein the Flt1 agonist is a VEGF A Flt1 selective agent.

4. The method of claim 1, wherein the Flt1 agonist is VEGF A, PlGF or VEGFB.

5. The method of claim 1, wherein the Flt1 agonist is a small molecule agonist of Flt1.

6. The method of claim 1, wherein the renal disease is characterized by a decrease in VEGF levels.

7. The method of claim 1, wherein the renal disease is inflammatory kidney disease.

8. The method of claim 7, wherein the inflammatory kidney disease is characterized by alterations in inflammatory cells, immune complex depositions or complement activation in affected glomeruli.

9. The method of claim 8 wherein the immune complex deposition is IgM deposition.

10. The method of claim 8, wherein the complement activation comprises activation of C1q, C3 and C4.

11. The method of claim 1, wherein the renal disease comprises glomerulonephritis (renal failure).

12. The method of claim 11, wherein the glomerulonephritis is determined by proteinuria, glomerular sclerosis, or hypertension.

13. The method of claim 12, wherein the glomerulonephritis is determined by decreased survival of kidney mesangial cells, an increase in gene expression of ECM synthesis or a reduction in matrix degradation.

14. The method of claim 11, wherein the glomerulonephritis is focal segmental glomerulosclerosis (FSGS).

15. The method of claim 1, further comprising administering an effective amount of a second agent, wherein the second agent is an angiogenic agent.

16. The method of claim 1, further comprising administering an effective amount of a second agent, wherein the second agent is a second Flt1 agonist.

17. The method of claim 15, wherein the angiogenic agent is VEGF.

18. The method of claim 2, wherein the antibody is a monoclonal antibody.

19. The method of claim 2, wherein the antibody is a chimeric, humanized or human antibody.

20. The method of claim 1, wherein the subject has an infection causing the renal disease.
